(19) United States
(12) Patent Application Publication
(10) Pub. No.: US 2013/0096067 A1
(43) Pub. Date: Apr. 18, 2013

(54) DROSOPHILA TUMOR STEM CELL MODEL AND USES THEREOF

(71) Applicants: Norbert Perrimon, Arlington, MA (US); Michele Markstein, Amherst, MA (US)

(72) Inventors: Norbert Perrimon, Arlington, MA (US); Michele Markstein, Amherst, MA (US)

(21) Appl. No.: 13/617,049

(22) Filed: Sep. 14, 2012

Related U.S. Application Data
(60) Provisional application No. 61/534,667, filed on Sep. 14, 2011.

(51) Int. Cl. A01K 67/033

(52) U.S. Cl. CPC A01K 67/0339
USPC 514/20.1; 800/13; 800/3; 435/8

(57) ABSTRACT

The present invention provides a transgenic Drosophila tumor stem cell model and uses thereof. Also provided is a method to selectively inhibit cancer stem cells in a mammal having refractory intestinal cancer or a relapse of intestinal cancer.

### CROSS-REFERENCE TO RELATED APPLICATIONS

The present application claims the benefit of the filing date of U.S. application Ser. No. 61/534,667, filed on Sep. 14, 2011, the disclosure of which is incorporated by reference herein.

### BACKGROUND

The Drosophila gut has emerged in recent years as a simple model system to study the biology of mammalian stem cells (Casali et al., 2009). For example, as in mammals, fly intestinal stem cells (ISCs) are multipotent and divide about once a day to maintain themselves and to generate daughter cells that differentiate into two classes of cells: large absorptive cells called enterocytes (ECs) and a heterogeneous population of secretory cells called enteroendocrine cells (Micchelli et al., 2006; Ohlstein et al. 2006). In addition, Drosophila and mammalian ISCs rely on the Wnt/Wg and Notch pathways to regulate their self-renewal and differentiation (Casali et al., 2009). Importantly, mutations in these pathways can generate stem cell-initiated tumors in both the Drosophila and mammalian gut (Lee et al., 2009; Barker et al., 2009; Fre et al., 2005; Stanger et al., 2005; Lin et al., 2008).

In addition to the Notch and Wnt pathways, Jiang et al. (2011) and Biteau et al. (2011) found that one of the pathways most commonly associated with human cancers, the Ras/MAPK pathway (Downward, 2003), is active in Drosophila ISCs. Mutations in the Ras/MAPK pathway are associated with 50-60% of human colorectal cancers: 40-50% of them have activating mutations in RAS, while another 10% have mutations in B-RAF (Rajagopalan et al., 2002).

### SUMMARY OF THE INVENTION

The present invention provides for a transgenic Drosophila tumor stem cell model. In one embodiment, the invention provides a transgenic Drosophila strain that allows for regulatable, e.g., inducible, expression of an oncogene in stem cells, such as intestinal stem cells (ISCs), thereby providing a tumor stem cell (TSC) model and an in vivo system for drug screening. In one embodiment, the present invention may provide a system to study cellular and molecular aspects of ISCs under both normal homeostatic and aberrant oncogenic conditions because Drosophila ISCs hold many parallels to their mammalian counterparts. Although a transgenic Drosophila ISC model is described in detail below, the system is applicable to the study of other stem cells and other cancers, e.g., transgenic fly models that express one or more oncogenes in stem cells other than intestinal stem cells can be prepared. Moreover, transgenic flies that express signal proteins involved in cell proliferation and propagation that are shared between flies and humans (e.g., Notch) may provide for other fly models useful to understand mammalian cell, e.g., stem cell, proliferation and propagation under both normal and aberrant conditions.

In one embodiment, the invention provides a transgenic Drosophila having a plurality of expression cassettes, e.g., stably integrated expression cassettes. In one embodiment, a first expression cassette comprises a tissue- or cell lineage-specific transcription regulatory element operably linked to an open reading frame encoding a gene product that regulates transcription of a specific transcription regulatory element in trans, e.g., by binding that element (a “driver”). For instance, the driver may be a transcription factor including one that is modified, e.g., truncated or substituted or fused to another molecule, relative to a wild-type transcription factor. In one embodiment, the driver does not regulate transcription regulatory elements in the Drosophila genome (that is, the driver is a heterologous transcription factor or may be a modified Drosophila transcription factor that does not bind endogenous (native) Drosophila genes).

Another expression cassette in the plurality of expression cassettes, e.g., a second expression cassette, comprises a transcription regulatory element that includes a nucleotide sequence that binds the driver, which is operably linked to an open reading frame encoding an oncogene. Oncogene expression is generally lethal and so the present system provides for upregulation of oncogene expression via the binding of the driver to the nucleotide sequence that binds the driver, which is linked (5′) to the oncogene. A further expression cassette, for instance, a third expression cassette, comprises a transcription regulatory element that includes a nucleotide sequence that binds the driver, e.g., a nucleotide sequence that is identical to that in the second expression cassette, which is operably linked to an open reading frame for a first marker gene, e.g., one that is optically detectable (e.g., detectable by colorimetric, fluorescent or luminescent methods). Optically detectable gene products (“reporters” or “markers”) useful in the invention include, but are not limited to, fluorescent proteins, for instance, green fluorescent protein, yellow fluorescent protein or red fluorescent protein, luminescent proteins such as luciferase, photoproteins such as Aequorin photoprotein or obelin photoprotein, chloramphenical acetyltransferase, a phosphatase such as alkaline phosphatase, a peroxidase such as horseradish peroxidase, beta-galactosidase, beta-lactamase or beta-glucuronidase. Other marker genes that may be employed include, but are not limited to, gene sequences that encode for resistance to antibiotics such as ampicillin, tetracycline, kanamycin, puromycin, bleomycin, streptomycin, hygromycin, neomycin, Zeocin™, and the like, a bleomycin-resistance gene, a metallothionein gene, a hygromycin B-phosphotransferase gene, the AUR1 gene, an adenosine deaminase gene, an aminoglycoside phosphotransferase gene, a dihydrofolate reductase gene, a thymidine kinase gene, a xanthine-guanine phosphoribosyltransferase gene, a hisD gene, a mprt gene, a bar gene, a nitrilase gene, a galactopyranoside gene, a xylosidase gene, an arabinosidase gene, a mutant acetolactate synthase gene (ALS) or acetoacid synthase gene (AAS), a methotrexate-resistant dhfr gene, a dalapon dehalogenase gene, a mutated anthranilate synthase gene that confers resistance to 5-methyl tryptophan, an R-locus gene, a xylE gene, an alpha-amylase gene, or a tyrosinase gene.

Another one of the expression cassettes (e.g., a fourth expression cassette) may comprise a transcription regulatory element that includes a sequence that binds the driver, e.g., a nucleotide sequence that is identical to that in the second expression cassette or the third expression cassette, or both, which is operably linked to an open reading frame for a second marker gene which is different than the first marker gene. In one embodiment, the second marker gene is detectable by colorimetric, fluorescent or luminescent methods.

A fifth expression cassette may comprise a transcription regulatory element that is not regulated by the driver but which is operably linked to an open reading frame encoding a gene product that regulates the driver or the expression thereof, for instance, a conditional mutant of a competitor or repressor of the driver. In one embodiment, the transcription regulatory element linked to the gene product that regulates the driver or the expression thereof comprises a constitutively expressed promoter. In other embodiments, the transcription regulatory element linked to the gene product that regulates the driver or the expression thereof may include tissue- or cell lineage-specific transcription control elements. In one embodiment, the gene product that regulates the driver or the expression thereof is a conditional mutant that is a temperature sensitive mutant, e.g., one that is stable at room temperature and so inhibits the activity of the driver at room temperature, e.g., at about 22° C. to about 27° C., but is unstable at higher temperatures. Thus, at a higher temperature, the driver is free to bind the transcription regulatory elements linked to the oncogene and the two different marker genes, allowing for their expression.

In another embodiment, the system provides for temporal (conditional) control of a driver, e.g., Gal4, by fusing the driver to a hormone receptor. In that embodiment, the driver is active, for instance, only binds the sequence that is 5′ to the reporter(s) and oncogene(s), upon the addition of hormone, e.g., estrogen or progesterone. Thus, in this embodiment four expression cassettes allow for the spatial and temporal control of reporter gene(s) and oncogene(s).

In another embodiment, the system provides for temporal control of a driver such as Gal4 by separately expressing portions of the driver (for instance, a “split” Gal4). In one embodiment, Gal4 is split into two portions and Gal4 is active only when both portions are present and capable of forming a transcriptional regulatory element that binds a specific DNA sequence, thereby regulating transcription of the reporter(s) and oncogene(s). In this embodiment, five expression cassettes allow for the spatial and temporal control of reporter gene(s) and oncogene(s). For example, one of the portions of the driver is expressed from a tissue- or cell lineage-specific transcription regulatory element and the other is expressed from an inducible transcription regulatory element, e.g., via exogenous agent addition or temperature change.

In yet another embodiment, the system provides for temporal control of a driver by introducing transcription inhibitory sequences between a sequence that binds the driver and a gene to be expressed. In one embodiment, the transcription inhibitory sequences may be flanked by sequences recognized by a DNA recombinase and a fifth expression cassette may be employed to express the DNA recombinase when expression of the gene(s) linked to the transcription inhibitory sequences is desired. In that embodiment, expression of the driver may be constitutive.

In yet a further embodiment, the system provides for temporal control of a driver by introducing transcription inhibitory sequences between a transcription regulatory element and an open reading frame for the driver. For example, the transcription inhibitory sequences may be flanked by sequences recognized by a DNA recombinase and a fifth expression cassette may be employed to express the DNA recombinase when expression of the driver is desired. In this embodiment, the transcription regulatory element for the driver may provide for constitutive expression.

In one embodiment, the transgenic Drosophila of the invention has two, three, four or expression cassettes. In one embodiment, a transgenic Drosophila has the first, third, fourth and fifth expression cassettes described above and that fly is crossed to a transgenic Drosophila with the second expression cassette. In one embodiment, a transgenic Drosophila has the first, second, third and fifth expression cassettes described above and that fly is crossed to a transgenic Drosophila with the fourth expression cassette. In one embodiment, the transgenic Drosophila has the first, second, fourth and fifth expression cassettes described above and that fly is crossed to a transgenic Drosophila with the third expression cassette. In one embodiment, the transgenic Drosophila has the second, third, fourth and fifth expression cassettes described above and that fly is crossed to a transgenic Drosophila with the first expression cassette.

In one embodiment, to prepare a transgenic Drosophila having all five expression cassettes, a first transgenic line having two of the five expression cassettes is crossed to a second transgenic line having the other three expression cassettes. For example, a first transgenic line having the second expression cassette (the oncogene) and the third expression cassette (having one of the marker genes) is crossed to a transgenic line having the driver, the other marker gene and the repressor. Thus, the present system allows for maximal flexibility. For example, a stock with the driver, e.g., Gal4, a regulator of the driver (e.g., Gal80ts), and the reporter gene(s) can be crossed to stock with an oncogene, so as to test different oncogenes, or different combinations of oncogenes, in a given tissue. Likewise, a stock with an oncogene, the regulator, and the reporter gene(s) can be crossed to any driver stock, so as to test different cell types with a particular oncogene.

In one embodiment, a transgenic Drosophila has the first, third, and fourth expression cassettes described above, e.g., where the driver is operably linked to an inducible transcription regulatory element, e.g., a heat shock promoter or a hormone inducible promoter, and that fly is crossed to a transgenic Drosophila with the second expression cassette. In one embodiment, a transgenic Drosophila has the first, second, and third expression cassettes described above, for instance, where the driver is operably linked to an inducible transcription regulatory element, e.g., a heat shock promoter or a hormone inducible promoter, and that fly is crossed to a transgenic Drosophila with the fourth expression cassette. In one embodiment, the transgenic Drosophila has the first, second, and fourth expression cassettes described above, e.g., where the driver is operably linked to an inducible transcription regulatory element, e.g., a heat shock promoter or a hormone inducible promoter, and that fly is crossed to a transgenic Drosophila with the third expression cassette. In one embodiment, the transgenic Drosophila has the second, third, and fourth expression cassettes described above, for instance, where the driver is operably linked to an inducible transcription regulatory element, e.g., a heat shock promoter or a hormone inducible promoter, and that fly is crossed to a transgenic Drosophila with the first expression cassette.

The expression of the marker genes in the fly allow for detection of expression in cells that also express the driver. The use of two different markers may allow for detection of expression at the cell and/or tissue level in cells isolated from the fly, in homogenates and/or in flies (at the organismal level). For example, when a gut-specific promoter was linked to a driver gene, marker gene expression was detected in Drosophila ISCs. In particular, the use of a luciferase reporter, e.g., a firefly or a Renilla luciferase, as one of the markers makes for easier tumor detection for high-throughput applications. However, any reporter that provides for quantitative measurements, e.g., a fluorescent protein such as GFP, RFP or YFP, may be employed. In one embodiment, one or both of the markers have a wide dynamic range and/or low or no background when present in an expression cassette.

Because the Ras/MAPK pathway is one of the pathways most commonly associated with human cancers, MAPK signaling in the transgenic flies was detected. MAPK signaling in the Drosophila ISCs was detected with a phospho-specific antibody that recognizes the active di-phosphorylated form of ERK (dpERK), a downstream kinase that mediates MAPK signaling. dpERK was found in ISCs and their undifferentiated daughter cells (enteroblasts (EBs)), but not in differentiated daughter ECs or EEs. These results demonstrated that MAPK signaling was active in ISCs and their undifferentiated daughters, but is not actively maintained in differentiated cells.

The adult transgenic Drosophila of the invention was found to be an effective in vivo model to identify compounds targeting specific tumor stem cells (TSCs) as a result of the exquisite control over oncogene expression in certain cells, e.g., lineage specific stem cells, in the fly. In particular, the creation of an ISC tumor model that shares features with mammalian cancer stem cells (CSCs) provides for quantitative methods to screen adult flies for tumor inhibitors. Thus, as described herein, changes in intestinal tumor growth were detected by comparing luciferase activity specific for intestinal stem cell tumors as a result of the esg-Gal4 driver, in whole-animal extracts of control and drug-treated flies. This allowed for examination of tumor growth in animals in parallel using a 96-well multi-homogenizer, e.g., using 1 to 10 flies/well, for instance, 2 to 5 flies/well, rather than dissecting animals one-by-one. In every case examined, reductions in luciferase correlated with reductions in the size of the tumor. For example, treatment with the mitotic inhibitor colchicine eliminated virtually all of the tumor cells, and likewise resulted in a steep drop in luciferase activity compared to DMSO treated controls. The drop in luciferase activity was not as severe as the loss of intestinal tumor cells, however, because the luciferase signal in whole-animal extracts is derived not only from the intestine, but other tissues where the esg-Gal4 driver is active, most notably the salivary glands and the malpighian tubules. Nonetheless, luciferase activity and tumor size were largely correlative because the intestine contributes to the majority of the total luciferase activity, and because the intestine has better proximity to ingested drugs and therefore greater responsiveness to drugs than the other tissues.

Results from a screen of about 7,500 known bioactives showed that the transgenic fly could identify pathway-specific inhibitors as well as compounds known to be effective in humans. For example, in a screen of about 550 compounds, MAPK, TOR, and Notch inhibitors were identified as potent anti-TSC compounds. Moreover, the present assay can distinguish Notch inhibitors from cytotoxic drugs and most other drugs because these inhibitors result in an increase in total luciferase activity rather than a decrease in total luciferase activity. In the absence of Notch signaling, stem cell daughters cannot differentiate. Thus, stem cells only give rise to stem cells, leading to an increase in cells expressing the driver which in turn drives expression of the marker(s) (e.g., esg-Gal4>UAS-luciferase). The Raf(gof) genetic background is particularly suited to respond to Notch inhibitors, and those inhibitors may be useful to inhibit or treat many kinds of cancer including colorectal cancer and leukemias, and may be useful to enhance stem cell numbers in diseases other than cancer.

Accordingly, the invention provides a method to screen compounds. The method includes providing a transgenic Drosophila of the invention which expresses the transcription factor under conditions that allow for binding of the transcription factor to the nucleotide sequence, and exposing the transgenic Drosophila to one or more compounds. In one embodiment, the expression of the transcription factor is induced prior to exposing the transgenic Drosophila to the one or more compounds. In one embodiment, the expression of the transcription factor is induced after to exposing the transgenic Drosophila to the one or more compounds. In one embodiment, the expression of the transcription factor is induced concurrently with the exposure of the transgenic Drosophila to the one or more compounds. At least one of the optically detectable gene products is detected in or in a portion of the transgenic Drosophila. An alteration in the presence or amount of the at least one optically detectable gene product in that transgenic Drosophila relative to the presence or amount of the corresponding optically detectable gene product in a corresponding control transgenic Drosophila that is not exposed to the one or more compounds or a portion of the control transgenic Drosophila, is indicative of a compound that alters the number of stem cells.

It was surprising that several FDA-approved anti-cancer drugs actively promoted wild-type stem cell proliferation. This effect was mediated by evolutionarily-conserved JAK-STAT cytokines from the stem cell microenvironment, indicating that similar effects might occur in humans and underscoring the importance of using in vivo models like Drosophila for anti-CSC drug evaluation. A wide variety of FDA approved anti-cancer drugs can affect the stem cell niche. In response to these drugs, the niche activates the JNK stress pathway and expresses IL6-related cytokines which activate JAK-STAT signaling in the stem cells, thereby inducing the stem cells to overproliferate. To specifically detect drugs with these affects, drugs were fed to JNK and JAK-STAT reporter flies, that were otherwise wild-type and lacked any oncogenic cassettes. The JNK reporter flies carry a puc-LacZ transcriptional sensor of the JNK signaling pathway as well as an esg-Gal4; UAS-GFP cassette to visualize the intestinal stem cells. The Jak-Stat reporter flies carry a GFP-tagged secreted ligand, UPD3-GFP, that is a homologue of IL-6 and that activates the Jak-Stat pathway. The drugs were fed to flies for three days and then screened with three reporters: a puc-LacZ reporter to detect JNK signaling in the niche, and an esg-Gal4; UAS-GFP reporter to detect whether the stem cells have hyperproliferated. Therefore, the invention also provides an in vivo assay to identify compounds that can provoke wild-type stem cells to hyperproliferate by inducing stress in stem cell daughter cells. Compounds that provoke this response may be considered potentially more deleterious than compounds that do not provoke this response.

The invention provides a method to selectively inhibit cancer stem cells in a mammal having refractory intestinal cancer or a relapse of intestinal cancer. The method includes comprising selecting a compound that alters the activity of intestinal tumor stem cells but not wild-type stem cells; and administering to the mammal an effective amount of the compound. In one embodiment, the compound may be methotrexate, dactinomycin, gemcitabine, triptolide, vinblastine, floxuridine, vincristine, bortezomib, tunicamycin, paclitaxel, mitomycin C, okadaic acid, camptothecin, topotecan, cycloheximide, tamoxifen, triethylenemelamine, thiotepa, daunorubicin, ICRF-193, calyculin A, aristolochic acid, rapamycin, actinomycin D, alamethicin, oligomycin A, latrunculin B, piceatannol, 10-hydroxycamptothecin, 5-iodotubercidin or compound E.

Therefore, the present system is applicable to the study of a wide variety of cancers using one or more different oncogenes that are temporally and/or spatially expressed, e.g., in specific stem cells.

### BRIEF DESCRIPTION OF THE FIGURES

FIG. 1. Characterization of wild-type ISCs and Raf-1^{gof} stem cell tumors. A) Wild-type intestinal stem cells (ISCs) and their undifferentiated enteroblast (EB) daughters express esg (green). Many of the ISCs also stain positively for dpERK (red, top panel) and Delta (red, lower panel). The major class of differentiated daughter cells, enterocytes (EC), can be distinguished from their diploid progenitors and sisters by their large polyploid nuclei, evident by nuclear staining with DAPI (blue). B) Raf-1^{gof} stem cell-initiated tumors express dpERK (red, top panels) and Delta (red, lower panels). Throughout tumorigenesis a population of Delta positive “tumor stem cells” is maintained (lower panels). C) Optical cross-sections show that the intestine becomes transformed into multilayered disorganized tumor masses by day 3 of Raf-1^{gof} tumorigenesis, e.g., esg-expressing cells (green) and differentiated ECs (distinguished by large polyploid nuclei) occupy all tumor layers. Staining with the F-actin probe, phalloidin (red), marks the muscle layers surrounding the gut as well as the apical brush border of the ECs. D) Transplantation of GFP-labeled intestinal fragments into unlabeled hosts (injection site marked by yellow triangle) shows that wild-type intestines do not noticeably propagate in hosts (n=0/160 transplants), whereas fragments from Raf-1^{gof} day 3 tumors proliferate rapidly, as shown at 2 days and 7 days after injection in the same host animal (n=14/132 transplants).

FIG. 2. Luciferase-based screen of 550 known bioactives for tumor stem cell inhibitors. A-B) Treating flies with the mitotic inhibitor colchicine (which eliminates virtually all of the Raf-1^{gof} tumor cells), can be either visualized by the loss of GFP-expressing cells in dissected guts (A), or quantified in by loss of luciferase activity in whole-fly lysates (B). C) Luciferase was measured from the intestines and bodies of individually dissected flies with Raf-1^{gof} three day tumors. On average the intestine contributes about two-thirds of the total luciferase (n=7). D) Summary of luciferase activity results from a screen of 568 known bioactives, 15 of which were FDA approved drugs. Luciferase activity was averaged from multiple wells for each compound when available and the activity is shown as the log₂ of the fold difference relative to DMSO controls. E) Pathway-specific compounds and broadly cytotoxic compounds identified by the screen were confirmed by replicate luciferase assays, shown as activity relative to DMSO controls (see FIG. 4). Error bars denote one standard deviation from the mean; for all samples, p<0.001 based on non-parametric Rank-Sum analysis.

FIG. 3. Tumor stem cells (TSCs) are mitotically active and required throughout tumorigenesis. A) Staining of Raf-1^{gof} cells at day 1 and 4 with pH3 and DAPI. B) Raf-1^{gof} cells treated with DMSO, vinblastine or vincristine at day 0-1 or 3-4.

FIG. 4. Replicate luciferase assays confirm pathway-specific and FDA-approved hits from the screen.

FIG. 5. Pathway-specific compounds confirmed by RNAi. A) Raf-1^{gof} TSC flies were treated for three days with 5-iodotubercidin, a MEK inhibitor, rapamycin, a TOR inhibitor, or Compound E, a Notch inhibitor. Control flies were treated with DMSO. An esg-GFP reporter marks tumor cells generally (green, upper panel) and Delta staining specifically marks the TSCs (red, lower panel). B) The requirement for MEK and TOR signaling was confirmed by RNAi against Dsor, Drosophila MEK, and RNAi against Raptor, a required component of the rapamycin-sensitive TOR complex (called TORC1). The effect of compound E was confirmed by expressing a Notch Dominant-Negative transgene in Raf-1^{gof} TSCs.

FIG. 6. FDA-approved drugs identified by the screen are potent inhibitors of Raf-1^{gof} TSCs.

FIG. 7. FDA anti-cancer drugs that block TSCs can have differential effects on the microenvironment and growth of wild-type ISCs. A) The effects of three representative FDA-approved drugs, methotrexate, bortezomib, and paclitaxel, on Raf-1^{gof} tumors are shown. Flies were treated with DMSO alone as a control. All of the FDA drugs are potent inhibitors of Raf-1^{gof} tumors, as visualized by esg-GFP (green, upper panels) and by Delta staining of the same dissected guts (red, lower panels). B) The effect of each drug on wild-type guts was visualized with an esg-Gal4 reporter marking ISC/EBs progenitors (green) and a puc-LacZ reporter (red) for JNK signaling (red) in upper panels, and with an upd3-GFP reporter (green, lower panel) for JAK-STAT signaling in lower panels in EC cells, and by inducing an expansion of esg-expressing progenitor cells. C) The expansion of ISCs/EBs in response to bortezomib and paclitaxel is less severe in animals with a loss-of-function JAK allele, hop^{msv1}.

FIG. 8. Drosophila Raf-1^{gof} tumors require AKT signaling.

FIG. 9. FDA-approved drugs have differential effects on the microenvironment and growth of wild-type progenitors.

FIG. 10. Pathway-specific inhibitors have no effect on the growth of wild-type progenitors or the microenvironment.

### DETAILED DESCRIPTION OF THE INVENTION

Cancer stem cells (CSCs), or tumor-initiating cells, are a relatively new area of cancer research. CSCs are cells that self-renew and are thought to be able to differentiate into all cell types found in a tumor, making them tumorigenic. Non-CSCs would be unable to differentiate into other cell types and so would not be tumorigenic. The implication is that if a cancer treatment does not eliminate all CSCs, those CSCs will be able to form new tumors. Thus, treatments that specifically target CSCs while leaving normal stem cells untouched will prevent tumor recurrence and further metastasis.

Usually, cancer treatment efficacy is measured by the size reduction in the tumor after treatment. However, this method ignores the significance of CSCs; a treatment could successfully kill off other tumor cells and reduce the tumor size, but leave the CSCs alive. Thus, a challenge in cancer therapeutics is to identify compounds that can kill cancer stem cells.

To find drugs that target CSCs, a new method of gauging cancer drug efficacy is necessary because these drugs could either be killing off CSCs or causing their differentiation. The challenge of finding anti-CSC drugs has been focused on developing methods to culture CSCs (Gupta et al., 2009). However, since stem cell biology is highly dependent on the stem cell microenvironment (Morrison, 2008; Voog et al., 2010), finding effective anti-CSC drugs also demands in vivo approaches that preserve the microenvironment.

Exemplary Drosophila TSC Model and Methods to Prepare the Transgenic Fly

The invention provides a transgenic Drosophila that is a CSC (TSC) model. The presently described Drosophila model is particularly useful to screen for drugs that may alter human TSCs, e.g., when there are similar pathways of self-renewal in fly and human stem cells. In particular, the present invention provides a system to screen for drugs in vivo that reduce tumor growth and in particular CSC in a tissue- or cell lineage-specific manner. For example, using transgenic fly models for CSCs, flies are fed drugs, e.g., in 96-well plates. The flies express an optically detectable gene product that correlates with tumor size/tumor cell number so large numbers of drugs can be easily and quickly screened for their ability to reduce tumor size ro tumor cell numbers. For instance, imaging can be used to find drugs that reduce the size of the tumors, e.g., in the fly intestine. The present invention thus allows for high-throughput screening, while also preserving the intricacies of the in vivo system. In addition, the system does not rely on xenografts.

The transgenic fly comprises a plurality of expression cassettes. The expression cassettes may include any combination of the following cassettes: a first expression cassette having a regulatable or tissue- or cell lineage-specific promoter operably linked to an open reading frame for a heterologous transcription factor; a second expression cassette having a transcription regulatory element comprising a nucleotide sequence that binds the transcription factor and a promoter that are operably linked to an oncogene; a third expression cassette having a transcription regulatory element comprising a nucleotide sequence that binds the transcription factor and a promoter that are operably linked to a first marker gene that encodes a marker gene, such as an optically detectable gene product; a fourth expression cassette having a transcription regulatory element that is not regulated by the transcription factor but includes a promoter operably linked to an open reading frame encoding a repressor or competitor of the transcription factor that is a conditional mutant; and a fifth expression cassette having a transcription regulatory element comprising a nucleotide sequence that binds the transcription factor and a promoter that are operably linked to a second marker gene, e.g., one that encodes an optically detectable gene product. In one embodiment, the transgenic Drosophila comprises all five expression cassettes. In one embodiment, the transgenic Drosophila may be prepared by crossing a transgenic fly which comprises the first, third, fourth and fifth expression cassettes with one having the second expression cassette. The conditional mutant repressor or competitor may be a temperature sensitive mutant.

In one embodiment, the heterologous transcription factor comprises GAL4. The GAL4 system is one example of a bipartite system useful in TSC models in transgenic organisms. For example, one transgenic line, the driver, expresses GAL4 in a known temporal or spatial pattern and a second transgenic line, the responder, contains a upstream activation sequence (UAS)-dependent transgene. The GAL4 system was designed to generate a range of driver lines in which a diverse array of regulatory elements controlled GAL4 expression. About 900 Gal4 driver lines are available at the Bloomington Indiana Stock Center: http://flystocks.bio.indiana.edu/Browse/misc-browse/gal4.php. Exemplary drivers for stem cells in the fly female germline, male germline, somatic tissues of the female germline, intestine and the malpighian tubules include, but are not limited to, for the female germline: nos-Gal4(VP), ovo-Gal4, or otu-Gal4; male germline: Esg-Gal4 or Nos-Gal4; female somatic stem cells; C587-Gal4; Brain: Elav-Gal4 or Grainy head-Gal4; blood: serpent-Gal4 or dome-Gal4.

Other systems that may be useful in TSC models in transgenic organisms include the Q system, e.g., where QF is a transcriptional activator and QS represses QF (see, e.g., Potter et al., Cell, 141:536 (2010), the disclosure of which is incorporated by reference herein), and the LexA system, where LexA represses genes having certain upstream sequences until activated RecA promotes autocatalytic cleavage of LexA.

Separation of the GAL4 driver and the UAS responder offers several advantages. First, a single UAS-dependent transgene can be analyzed in multiple tissues and/or time-points through the use of different drivers. Second, as the UAS construct is effectively silent in the absence of GAL4, transgenics encoding toxic proteins such as ricin, apoptotic proteins or oncogenes can be generated without potentially deleterious effect.

Temporal control, without cell or tissue specificity, of GAL4 activity can be achieved by driving GAL4 expression from a heat-shock promoter. Another approach to regulate temporal expression of a UAS-responder is to make use of the yeast protein GAL80, which binds the transactivation domain of GAL4 and prevents GAL4 from activating transcription in yeast. GAL80 can repress GAL4 in Drosophila, and when expressed ubiquitously under the control of the tubulin 1α promoter, represses GAL4 activity in all tissues. In one embodiment, a temperature-sensitive version of the GAL80 protein (GAL80ts) is expressed ubiquitously under the control of the tubulin 1α promoter. In this embodiment, GAL80 repression of GAL4 is alleviated by a simple temperature shift.

Another approach to regulate temporal expression of a UAS-responder is a hormone inducible variant of GAL4. Two GAL4-hormone receptor chimeras are available: GAL4-estrogen receptor and is a fusion of GAL4-progesterone receptor and the activation domain of p65. Transcriptional activation in both systems requires the addition of the appropriate ligand as both are transcriptionally silent until bound by the ligand. The onset of expression is controlled by feeding the fly or larva the ligand at an appropriate time. The system is reversible by removal of the fly or larva from feed containing the ligand.

One approach to refining temporal regulation of the GAL4 system is to combine it with the FLP-out technique. For example, a terminator cassette flanked by FLP recognition target (FRT) sites can be placed between the UAS promoter and the gene to be expressed, rendering the transgene silent. Transgene by expression of the FLP recombinase which removes the terminator cassette. Use of a heat shock inducible FLP recombinase (hs-FLP) affords temporal control to the onset of transgene expression. A similar result can be achieved by placing a FRT-flanked terminator cassette in front of the GAL4-coding sequences. In addition, it is possible to use other characterized promoters to drive FLP expression in a spatiotemporal fashion.

Another approach is the use of tetracycline. Two versions of the tetracycline system exists: Tet-On, in which the addition of the drug results in an active reverse-tetR (rtTA) causing transgene activation from the tet operator, and Tet-Off, in which addition of the drug inactivates tTA, and in turn, expression from the tet operator is switched off.

A second binary system, which functions in an analogous manner to the GAL4 system, is based on the bacterial lexA protein. Combining the GAL4 and lexA systems may facilitate more precise cell labeling. In another system, the separate DNA binding domain (DBD) and activation domain (AD) of GAL4 are fused to a heterodimerizing leucine zipper motif and each fusion protein is expressed separately. Only when they are present in the same cell can the leucine zippers direct heterodimerization, resulting in the formation of a functional activator. The fusion proteins, ZIP::GAL4 DBD and GAL4AD::ZIP (or a second activation domain VP16AD::ZIP), are transcriptionally inactive. The inactive constructs are referred to as “hemi-drivers.” Transactivation requires the reconstitution of GAL4 by the presence of both ZIP::GAL4 DBD and GAL4AD::ZIP, which will only occur in the domains of overlapping expression of the hemi-drivers. Coexpression of the DBD and AD fusion proteins results in expression of a UAS-GFP reporter.

In one embodiment, a first Drosophila line (line 1) has one or more reporters for high-throughput quantitative measurements of tumor growth and conditional control of the expression of the reporters and oncogene, and line 2 has the oncogene, e.g., the Gal4-inducible constitutively active human UAS-Raf1(gof). The conditional control is provided by tissue-, cell lineage- or cell-type specific expression of a transcription factor that does not regulate endogenous fly genes, and constitutive expression of a conditional, e.g., mutant, repressor of the transcription factor. For example, for a ISC model a “gut-specific” promoter drives expression of GAL4. GAL4 does not turn on endogenous fly genes; it only turns on genes with Yeast Upstream Activating Sequence (“UAS”) sites. Thus, in one embodiment, after crossing line 1 and line 2, progeny have transgene 1 (Esg>Gal4), transgene 2 (UAS>oncogene), e.g., human Raf(gof) or myr-AKT or Notch(DN), transgene 3 (UAS>luciferase), transgene 4 (UAS>GFP), and transgene 5 (Tubulin>Gal80(ts). Tubulin>Gal80(ts) is temperature-sensitive repressor of the Gal4 transcription factor that is ubiquitously expressed by the tubulin promoter. At room temperature, Gal80(fs) is functional and represses Gal4 activity. Thus, none of the Gal4 UAS-targets are activated at room temperature. When the temperature is raised, e.g., to 29° C., the complex with Gal80(ts) falls apart, allowing Gal4 to bind to each UAS, which results in the oncogene, luciferase, and GFP becoming expressed. Although many UAS-luciferase transgenes are integrated at loci randomly distributed across the fly genome, some UAS-transgenes are more sensitive than others for detecting changes in the gut, e.g., one at a location on the third chromosome, called attP2.

Exemplary oncogenes for use in the flies include but are not limited to those in the EGFR-Pathway, e.g., UAS-Raf1 (gof), UAS-RasV12, or UAS-EGFR activated; the Insulin/PI3K pathway, e.g., UAS-Insulin activated, UAS-myr-AKT, or UAS-PTEN-RNAi; the TGF-B pathway, e.g., UAS-Tkv activated; UAS-Myc; the Hippo pathway, e.g., UAS-hippo-RNAi, UAS-warts-RNAi, or UAS-Yki activated; the Wnt pathway, e.g., UAS-arm* or UAS-APC-RNAi; the Notch pathway, e.g., UAS-NotchDN; the Hedgehog pathway, e.g., UAS-Ci activated; the Jak-Stat pathway, e.g., UAS-hopTumL; or the JNK pathway, e.g., UAS-Puc-RNAi, or UAS-hep activated. To prepare metastatic tumor models, pathway activators may be combined with transgenes that interfere with cell polarity. For instance, flies with at least one oncogene transgene may be combined with a cell-polarity disrupting transgene, e.g., E-cadherin-RNAi, Crumbs-RNAi, PKC-RNAi, Lgl-RNAi, Scrib-RNAi, or Par6-RNAi.

For stem cell specific tumor models, combining UAS-RAF with UAS-Notch(Dominant Negative) was found to rapidly create growing tumors of relatively pure stem cells; that is, stem cells that cannot differentiate. Thus, Notch(DN) may be combined with other growth promoting transgenes to create additional stem-cell only tumors, but with distinct features and/or genetic requirements. Thus, combining Notch(DN) with a growth promoting transgene, such as those disclosed above, is envisioned.

The flies of the invention may be employed to screen compounds. The transgenic Drosophila of the invention is exposed to one or more compounds before, during or after, or any combination thereof, the transcription factor is expressed. The presence or amount of at least one of the marker genes is detected in the transgenic Drosophila or in a portion of the transgenic Drosophila. An alteration in the presence or amount of the marker gene relative to the presence or amount of the marker gene in a corresponding control transgenic Drosophila (that are not exposed to the one or more compounds) or a portion of the control transgenic Drosophila, is indicative of a compound that alters cancer stem cell numbers. In one embodiment, the one or more compounds are ingested by the transgenic Drosophila. In one embodiment, a “dose” range study may be conducted where specific amounts of a drug can be fed to the transgenic flies. The presence or amount of the one of the marker genes may be detected in specific cells in homogenates or in the organism. A compound may alter the number of tumor stem cells but not wild-type stem cells. A compound may enhance proliferation (number) of wild-type stem cells. The compound may induce the JNK and JAK-STAT pathways.

Exemplary ISC Tumor Model

The presently described Drosophila model is particularly useful to screen for drugs that may alter human TSCs, e.g., when there are similar pathways of self-renewal in fly and human stem cells. One such pathway is in the intestine, as described in more detail below. In humans, colon cancer originates in the epithelial cells as a result of mutations with the Wnt-APC-beta catenin signaling pathway. Other signals involved in self-renewal such as Hedgehog and Notch can also be part of cancer development, as the tumor-initiating cells must be able to divide when necessary and still stay dedifferentiated.

In mammals, the inner lining of the intestinal tube is a monostratified epithelium folded into millions of invaginations known as crypts. The small intestine of an adult mouse contains roughly a million crypts, each producing around 300 cells per day, every day of the mouse's life. This remarkable yield is ultimately sustained by a small population of stem cells (four to six cells) that reside at the base of each crypt. The progeny of mammalian intestinal stem cells (ISCs) does not differentiate immediately, but rather it is amplified by cell division during a process of continuous upward migration along the crypt axis.

The location and the precise identity of mammalian ISCs have been controversial issues due to the lack of specific marker genes and assays to study their properties (Barker et al., 2008b). In a decisive work, Hans Clevers and colleagues have recently identified bona fide gastrointestinal stem cells at the bottommost positions of the stomach, small intestine, and colon crypts (Barker et al., 2007). ISCs in the intestinal tract can be specifically recognized by the expression of Lgr5, a Wnt target gene that codifies for an orphan G protein coupled receptor of unknown function. Lgr5+ ISCs are multipotent, divide approximately once every day, and are capable of regenerating the intestinal epithelium for long periods (>12 months).

The presence of regenerative cells in the intestine of some arthropods was reported more than century ago, but it has not been until recently that Drosophila intestinal stem cells were revealed. The Spradling and Perrimon labs described the nature of adult Drosophila midgut ISCs (mgISCs) (Micchelli and Perrimon, 2006; Ohlstein and Spradling, 2006), while the Hartenstein group identified ISCs in the hindgut (hgISCs) (Takashima et al., 2008). The lining of the fruit fly midgut (i.e., the equivalent of the mammalian small intestine) is a pseudostratified epithelium composed mostly of large polyploid cells of absorptive function (enterocytes or EC) intermingled with two less abundant populations of diploid cells, enteroendocrine (EE) cells and midgut stem cells (mgISCs). mgISCs sit in basal position relative to the rest of epithelial cell types and show a wedge-like morphology that to some extent resembles that of mouse Lgr5+ cells. Unlike in mammals, mgISCs are the only known cell type in the posterior midgut that proliferates, as their progeny is not further amplified. Upon cell division, the descendants of mgISCs regenerate the stem cell pool and/or become quiescent progenitor cells (known as enteroblasts or EB cells), which ultimately differentiate to EC or EE cells (Micchelli and Perrimon, 2006; Ohlstein and Spradling, 2006).

The Drosophila midgut arises from the endoderm, like the mammalian intestinal tract. However, the hindgut, which is the anatomical equivalent of the mammalian large intestine, develops from ectodermal imaginal discs. In Drosophila intestinal cells, Notch signaling is used in a fashion similar to that of their mammalian counterparts, albeit with some notable differences. Midgut ISCs, EBs, and early-differentiated cells all express Notch receptor, but Delta, a ligand for Notch, is only present in mgISCs (Ohlstein and Spradling, 2006, 2007). Lack of Delta in the mgISCs produces tumor-like expansion of cells that resemble either ISCs or EE cells. On the other hand, constitutive Notch activation in mgISC results in reduced proliferation and premature differentiation toward an EC fate (Ohlstein and Spradling, 2007; Micchelli and Perrimon, 2006). These observations suggest that Delta expression in mgISC induces Notch signaling in daughter cells, which in turn triggers the differentiation process. Therefore, lineage selection and differentiation do not seem to rely on supporting cells or surrounding tissues.

Notch signaling seems to have opposite outputs regarding self-renewal in the mammalian and Drosophila intestinal epitheliums. Genetic or pharmacological blockade of Notch activity in mice causes the depletion of the progenitor cell compartment by promoting differentiation (van Es et al., 2005). On the contrary, reduction of Notch signaling in Drosophila intestine induces overgrowth of mgISCs due to impaired differentiation (Ohlstein and Spradling, 2006, 2007; Micchelli and Perrimon, 2006). Concordantly, excess of Notch signaling amplifies the number of progenitor cells in mammalian crypts (Fre et al., 2005), whereas it induces the differentiation of Drosophila mgISC (Ohlstein and Sprading, 2006, 2007; Michell and Perrimen, 2006).

Because of the parallels, Drosophila ISC models may be employed to screen drugs useful to prevent, inhibit or treat colon cancer. Colorectal cancer is the fourth most common form of cancer in the U.S., and the incidence of colon cancer increases with age and family history of cancer. In addition, inherited polyposis syndromes, such as familial adenomatous polyposis, put the patient at increased risk for colon cancer.

Colon cancer can take many years to develop, early detection greatly increases a patient's chances of survival. However, due to a number of factors (invasiveness of screening, low awareness), colon cancer screening rates are low. In addition, early-stage disease is almost always asymptomatic, unless the location of the tumor impedes defecation or causes bleeding. Surgery is an excellent treatment for early-stage colorectal cancer; the problem is finding it in time. Treatment for late-stage disease is rarely curative, as colon cancer is quite invasive and has likely metastasized. A treatment focused on the tumor-initiating cells would stop cancerous cells from proliferating early on and prevent initial microtumors from growing. That would especially be useful for patients who have polyposis syndromes, since they are at increased risk for colon cancer; such a drug could be taken regularly to stave off cancer development in one of their numerous polyps.

One non-limiting example of a transgenic Drosophila model, e.g., a transgenic ISC model that may be employed to screen drugs useful to prevent, inhibit or treat colon cancer, is described below.

### Example I

### Materials and Methods

### Drosophila Strains and Genetics

Controls: yw,yv;attP2 (Ni et al., 2008). Gal4 drivers and reporters: esg-Gal4,UAS-GFP,tub-Gal80^{ts} (Micchelli et al., 2006), UAS-luc2(luciferase at attP2) (Markstein et al., 2008), puc-lacZE69 (Ring et al., 1993), #42685}, upd3-Gal4 (Agaisse et al., 2003). UAS constructs: UAS-Raf-1^{gof} (Brand et al., 1994), UAS-raptor-RNAi (TRIP stock JF01088), UAS-Dsor-RNAi (TRIP stock JF01697), UAS-Akt-RNAi (TRIP stock HMS00007). UAS-NotchDN (Jacobsen 1998). Mutants: hop^{msv1} (Perrimon et al., 1986). Using the above stocks, an esg-Gal4 stock was created with both an inducible GFP reporter and an inducible Luciferase reporter: esg-Gal4,UAS-GFP,tub-Gal80^{ts}; UAS-luc2. For the chemical screens the following cross was performed to create Raf-1^{gof} intestinal tumors marked with GFP and luciferase: UAS-Raf-1^{gof} X esg-Gal4,UAS-GFP,tub-Gal80^{ts}; UAS-luc2.

### Antibodies

Primary antibodies: mouse monoclonal anti-Delta 1:50 (Developmental Studies Hybridoma Bank), rabbit monoclonal anti-dpERK 1:200 (Cell Signaling), rabbit polyclonal anti-β-galactosidase 1:10,000 (MP Biomedicals), rabbit polyclonal anti-phosphohistone H3 1:10,000 (Millipore). Secondary antibodies: Alexa 647-conjugated goat anti-rabbit secondary, Alexa 594-conjugated donkey anti-mouse secondary, Alexa 647-conjugated donkey anti-mouse secondary, Alexa 594-conjugated donkey anti-rabbit secondary, all 1:1000 (Molecular Probes). Alexa 660-conjugated Phalloidin 1:1000 (Molecular Probes)

### Immunofluorescence and Microscopy

Adult fly guts were dissected in PBS and fixed for 20 minutes as described (Lobo et al., 2007). Samples were incubated 1 hour in 5% NDS blocking solution, overnight in primary antibody, 1.5 hours in secondary antibody and 6 minutes in DAPI. They were rinsed and washed between steps in PBS containing 0.1% Triton X-100. Finally they were placed in mounting media (Vectashield) and mounted for microscopy. Images were taken with a Leica TCS SP2 confocal microscope.

### Drug Preparation

Primary screening drugs were provided by the Harvard Institute for Chemistry and Cell Biology (ICCB; 480 known bioactives library), and the National Cancer Institute (NCI) Drug Therapeutics Program (library of 88 FDA-approved oncology drugs). Secondary assays were conducted with freshly prepared 10 mM stocks that were diluted 1:100 in fly food, giving a final concentration of 100 μM. 5-iodotubercidin, Rapamycin, Compound E (Enzo Life Sciences), Methotrexate (Sigma), Actinomycin-D, Bleomycin, Bortezomib, Daunorubicin, Floxuridine, Gemcitabine, Irinotecan, Methotrexate, Mitomycin, Paclitaxel, TEM, Thiotepa, Topotecan, Vinblastine, and Vincristine (provided by the NCI).

### Drug Preparation in Fly Food

Drugs were dissolved in 100% DMSO. Drugs were mixed 1:100 with molten fly food at 37° C. and then aliquoted to either 5 mL tubes or 1 mL wells of a 96-well plate. The molten food was created by boiling an agarose-based food composed of: 2% yeast, 7% corn syrup, 1.5% agarose (composed of 1:11 parts standard agarose: low-melt agarose), made up in distilled water.

### Drug Screening Protocol

The drugs were screened in two stages. The ICCB known bioactives were screened in 5 mL round bottom vials. Flies were fed freshly prepared drugs (1 μL in 100 μL food) for three consecutive days. Flies were then put to sleep by CO₂ and aliquoted to the wells of a 96-well plate on ice. Each well contained 100 μL of glo lysis buffer (Promega E2661). Three flies were aliquoted to each well. Typically each drug was tested by 3-4 independent wells. The plates were stored at −20° C.

The FDA-approved drugs were screened in 1 mL deep 96-well plates. Flies were treated with a single dose of drug (3 μL in 300 μL food) for three days. The flies were then put to sleep by CO₂ and flipped to the wells of a standard 96-well plate on ice, with three flies per well. 100 μL of glo lysis buffer (Promega E2661) were then added to each well, and the plates were stored at −20° C. The FDA screen was independently repeated with a six-day protocol, such that flies were given a single dose of drugs for three days, and then another dose for three more days, before being harvested as above.

### Luciferase Assay

Flies were anesthetized and transferred in pools of 3 to 96-well plates containing 100 μL of glo lysis buffer (Promega E2661) and homogenized with a multiple homogenizer (Burkard Scientific BS00253) for 3-5 minutes. The plates were then centrifuged for 5 minutes at 4,000 rpm, and the lysates were then transferred to new 96-well “master” plates. Firefly luciferase assays were conducted with 20 μL of lysate as specified by Promega. Luminescence was counted using the Molecular Devices Analyst GT plate reader.

### Tumor Transplantation

Transplantation of tissue into adult hosts was performed as previously described (Caussinus et al., 2005) with minor modifications. One day after eclosion, esg>GFP (wild-type) and esg>GFP, Raf-1^{gof} flies were incubated at 29° C. for three days. Their guts were dissected in PBS, cut into small pieces, and loaded into a glass needle suitable for an Eppendorf FemtoJet Injection System. Tissue fragments were injected in the mid-ventral abdomen of young adult female w1118 host flies that were CO₂-anesthetized on a fly pad prior to transplantation. After the injection procedure the host flies were kept at 29° C. and tumor growth was monitored using a standard epifluorescence microscope.

### Results and Discussion

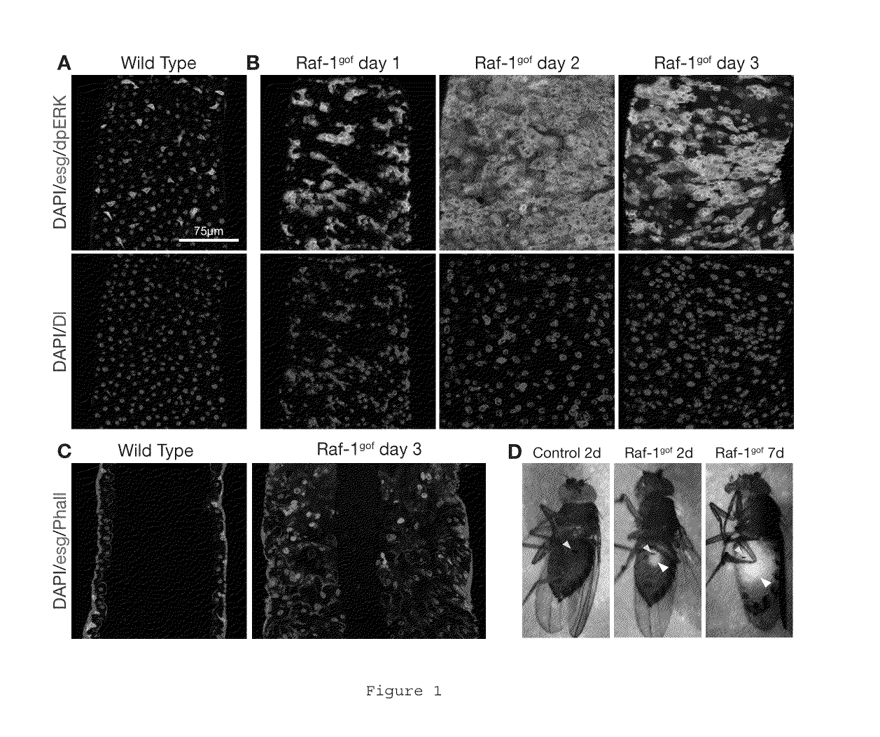

Since mutations in the MAPK pathway are prevalent in colorectal and other human cancers, it was determined whether hyperactivation of the pathway could produce stem cell-initiated tumors. Indeed, such tumors were produced by expressing a constitutively active human RAF-1^{gof} transgene (Brand et al., 1994) with the escargot (esg) Gal4 driver (Micchelli et al., 2006) which is active in ISCs and EBs (FIG. 1A). FIG. 1A shows that wild-type ISCs and their undifferentiated enteroblast (EB) daughters expressed esg (green), and were often found in pairs. Many of the ISCs also stained positively for dpERK (red, top panel) and Delta (red, lower panel). The major class of differentiated daughter cells, enterocytes (EC), could be distinguished from their diploid progenitors and sisters by their large polyploid nuclei, evident by nuclear staining with DAPI (blue, as shown in all figures).

Raf-1^{gof} stem cell-initiated tumors, created by expression of a human Raf-1^{gof} transgene in esg-expressing cells, began as clusters of largely stem cell-like cells (day 1) expressing dpERK (red, top panels in FIG. 1B) and Delta (red, lower panels in FIG. 1B), which grew to occupy the entire surface epithelium (day 2) and continued to differentiate (day 3). Throughout tumorigenesis, a population of Delta positive “tumor stem cells” was maintained (lower panels in FIG. 1B). Optical cross-sections of flies with Raf-1^{gof} stem cell-initiated tumors showed that the intestine, which is a monolayer in wild-type animals, became transformed into multilayered disorganized tumor masses by day 3 of Raf-1^{gof} tumorigenesis. Esg-expressing cells (green in FIG. 1C) and differentiated ECs (distinguished by large polyploid nuclei in FIG. 1C) occupied all tumor layers. Staining of those cross section with the F-actin probe, phalloidin (red in FIG. 1C), marked the muscle layers surrounding the gut as well as the apical brush border of the ECs. In wild-type guts the EC brush border is aligned with the gut lumen whereas in Raf-1^{gof} tumors it is highly disorganized.

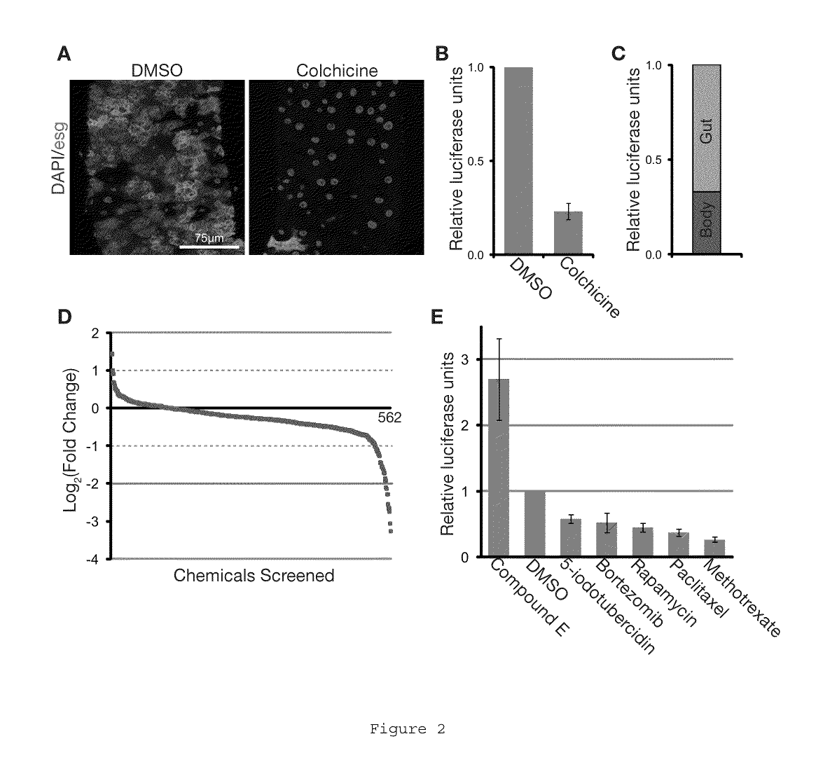

Thus, RAF-1^{gof} expression produced clusters of largely stem cell-like cells with strong dpERK staining that gave rise to multilayered heterogeneous tumors. As in mammalian cancers, many of the tumor cells aberrantly co-expressed differentiation and progenitor markers. For example, several cells with polyploid nuclei, a hallmark of terminal differentiation, simultaneously expressed the progenitor markers esg and dpERK. By day 3 of tumorigenesis, net growth of the tumors appeared to stabilize, with the bulk of the tumor composed of terminally differentiated polyploid cells (FIGS. 2B and 2C).

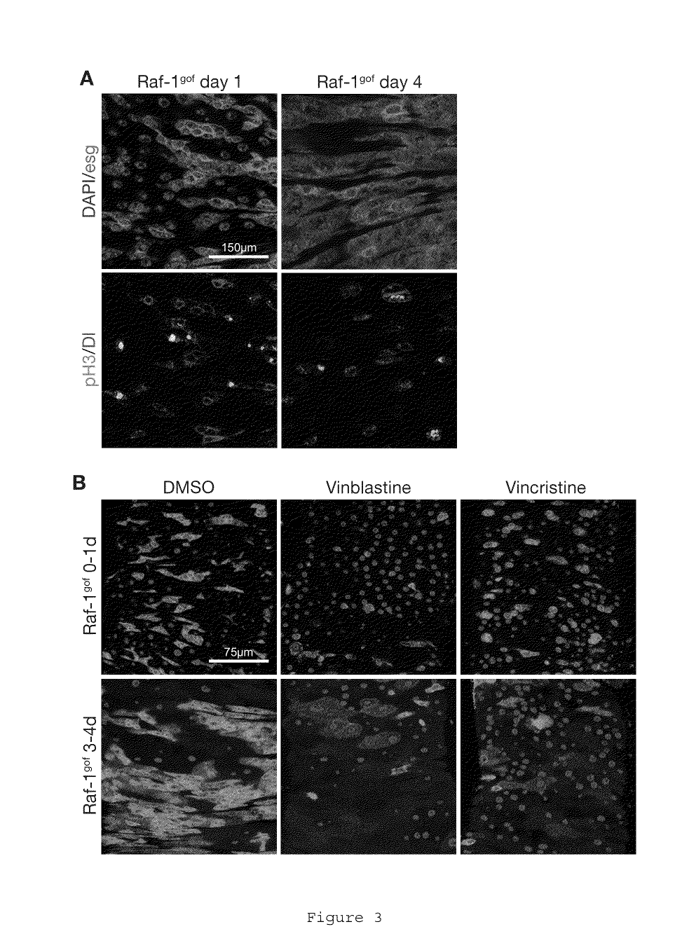

Moreover, strikingly, a subset of the Drosophila tumor cells retained stem cell-like characteristics, cells that are referred to as “tumor stem cells,” or TSCs. TSCs expressed the stem cell associated marker Delta (Ohlstein et al., 2007), a membrane bound ligand of the Notch pathway, and they were the only cells in the tumor found to be mitotically active, based on staining with antibodies to phosphorylated histone H3 (pH3) (FIG. 1B and FIG. 3A). As with mammalian CSCs, TSCs appeared to be both necessary and sufficient to maintain and propagate tumors. For example, tumors at any stage could be virtually eliminated by treatment with mitotic inhibitors, indicating that continued mitosis by TSCs is necessary to maintain the tumors (FIG. 3B).

Transplantation of GFP-labeled intestinal fragments into unlabeled hosts (injection site marked by yellow triangle in FIG. 1D) showed that wild-type intestines do not noticeably propagate in hosts (n=0/160 transplants), whereas fragments from Raf-1^{gof} day 3 tumors proliferate rapidly, as shown at 2 days and 7 days after injection in the same host animal (n=14/132 transplants), to occupy the entire abdomen of host animals, indicating that the mitotically active TSCs have regenerative ability. These results demonstrate that Raf-1^{gof} TSCs exhibit hallmarks of mammalian CSCs (Lobo et al., 2007): they are required to maintain the tumor and they have the capacity to propagate the tumor in transplantation experiments.

To exploit the TSC model for in vivo chemical screens, a high-throughput method was developed to track tumor growth, using a luciferase reporter assay instead of the conventional method of visualizing tumors with GFP. Luciferase is amenable to high-throughput screening (Markstein et al., 2008): it can be measured by simply homogenizing flies and measuring activity in a 96-well plate reader, a process that is considerably more efficient than hand dissecting animals one-by-one to visualize changes in GFP-expressing cells. The esg-Gal4 driver was employed to express luciferase in the TSCs and the effects of various compounds were compared by measuring luciferase in homogenates of control and drug-treated flies. In every case examined, reductions in luciferase correlated with reductions in the size of the tumor. For example, when flies were treated with the mitotic inhibitor colchicine, virtually all of the tumor cells were lost as well as about 80% of the luciferase activity (FIGS. 2A and 2B). The remaining luciferase activity stemmed from other tissues where the esg-Gal4 driver is active, such as the salivary gland and malpighian tubules (data not shown), which collectively contributed about 30% of the total luciferase activity (FIG. 2C). As these tissues were not as proximal to ingested drugs as the gut, it was unexpected that they would be as responsive to drugs. Indeed, this appears to be the case, as overall changes in esg-Gal4 expressed luciferase correlated well with changes in tumors of the gut.

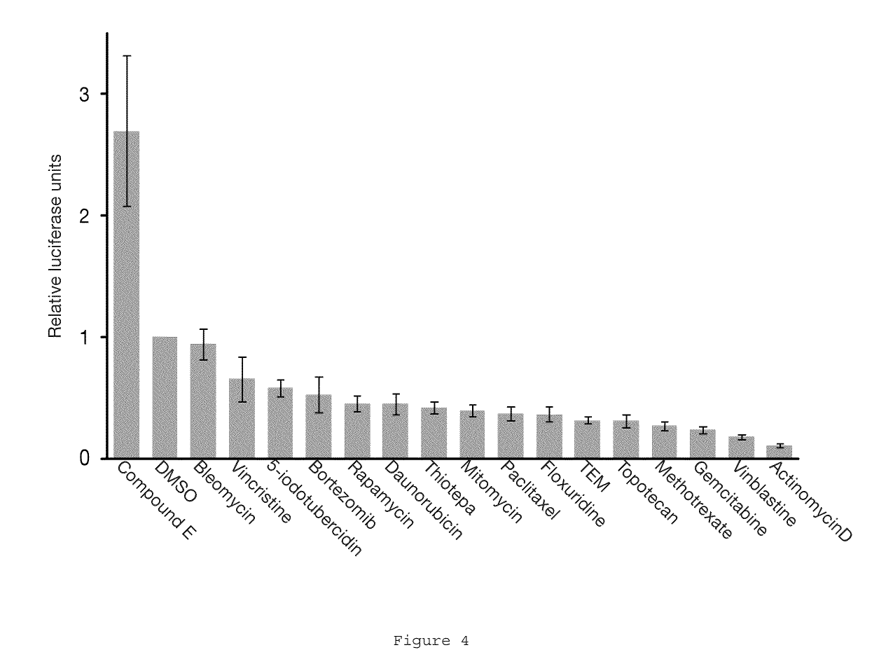

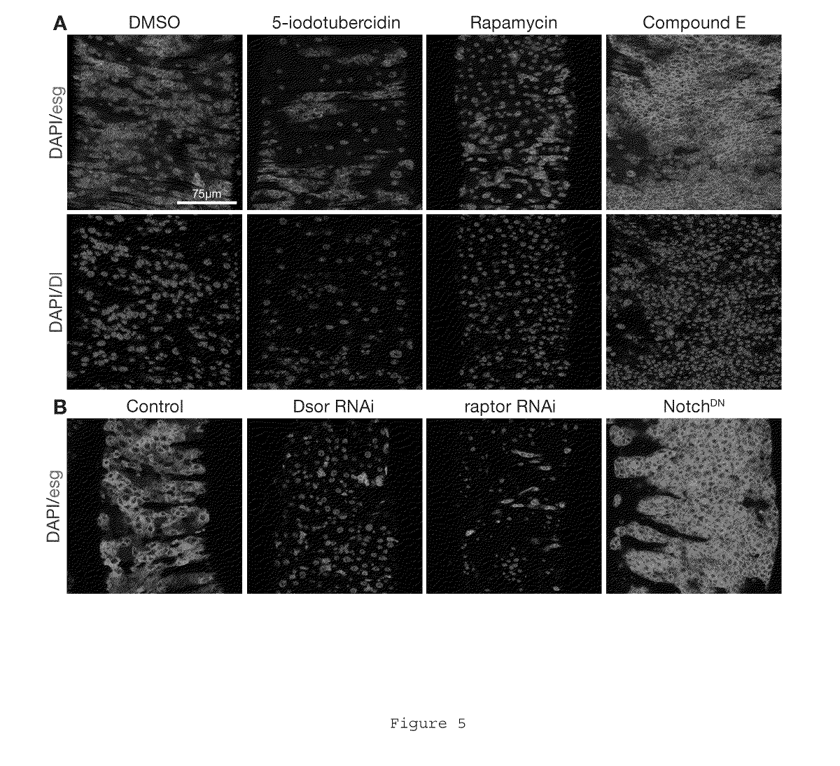

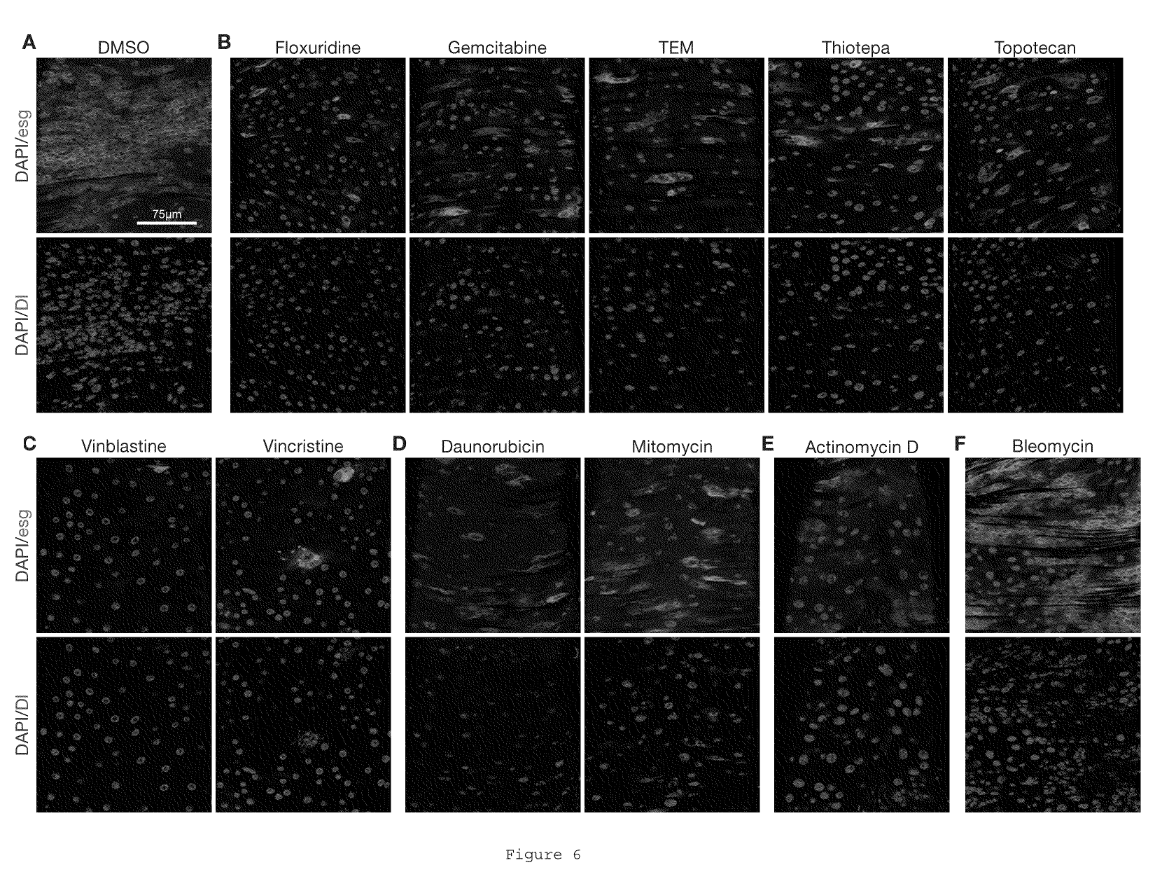

To determine what types of small molecules could be identified by the TSC model, the esg-Gal4 luciferase assay was employed to screen a library of about 550 known bioactives (FIG. 2D and Tables 1 and 2). Luciferase activity was averaged from multiple wells for each compound when available and the activity is shown as the log₂ of the fold difference relative to DMSO controls. The library included several pathway-specific inhibitors, as well as about 100 FDA-approved anti-cancer drugs. Using methods that were developed to effectively administer compounds to flies, compounds were fed to flies with one-day old Raf-1^{gof} tumors (FIG. 1B) and the effects on tumor growth assessed three days later. The screen identified 29 compounds that produced two-fold or greater changes (reduction) in luciferase activity (Table 3), 15 of which were FDA approved drugs. The drugs were grouped into the following classes: (1) pathway-specific compounds, (2) broadly cytotoxic compounds, best exemplified by FDA-approved anti-cancer drugs, and (3) compounds that affected the stem cell microenvironment. Compounds from each class were confirmed by replicate luciferase assays (FIG. 2E and FIG. 4) and by visualization of the GFP-expressing tumors in dissected guts (FIGS. 5, 6 and 7).

TABLE 1
Screen of 480 ICCB compounds in vials

| | | StDev | Log2 | |
|---|---|---|---|---|
| Drug Name | Fold | (fold) | (fold) | Proposed Role |
| methotrexate | 0.160 | 0.012 | −2.648 | DHFR inhibitor |
| triptolide | 0.170 | 0.022 | −2.553 | apoptosis inducer |
| vinblastine | 0.204 | 0.000 | −2.290 | tubulin inhibitor |
| tunicamycin | 0.263 | 0.000 | −1.925 | glycosylation inhibitor |
| paclitaxel | 0.274 | 0.000 | −1.870 | microtubule stabilizer |
| okadaic acid | 0.305 | 0.044 | −1.713 | PP1 PP2A inhibitor |
| camptothecin | 0.326 | 0.030 | −1.619 | topoisomerase 1 inhibitor |
| cycloheximide | 0.337 | 0.032 | −1.568 | protein synthesis inhibitor |
| tamoxifen | 0.339 | 0.000 | −1.562 | estrogen antagonist |
| ICRF-193 | 0.364 | 0.009 | −1.459 | topo II inhibitor that does |
| | | | | not cause DNA breaks |
| calyculin A | 0.413 | 0.000 | −1.276 | PP1, PP2a inhibitor |
| aristolochic acid | 0.422 | 0.030 | −1.243 | phospholipase A2 |
| | | | | inhibitor |
| rapamycin | 0.429 | 0.035 | −1.220 | FRAP inhibitor very |
| | | | | potent! |
| actinomycin D | 0.438 | 0.034 | −1.192 | transcription inhibitor |
| alamethicin | 0.445 | 0.000 | −1.167 | monovalent cation |
| | | | | ionophor |
| oligomycin A | 0.457 | 0.127 | −1.131 | Fo ATP synthase |
| | | | | inhibitor |
| mitomycin C | 0.470 | 0.045 | −1.088 | cross links DNA |
| Latrunculin B | 0.478 | 0.025 | −1.064 | actin inhibitor |
| Piceatannol | 0.493 | 0.094 | −1.020 | Syk inhibitor |
| 10-hydroxycamptothecin | 0.525 | 0.016 | −0.930 | topoisomerase 1 inhibitor |
| 5-iodotubercidin | 0.530 | 0.011 | −0.917 | ERK-2 inhibitor |
| staurosporine | 0.541 | 0.000 | −0.887 | kinase inhibitor |
| 2-methoxyantimycin A3 | 0.546 | 0.043 | −0.873 | Bcl-2/Bcl-XL ligand |
| | | | | induces apoptosis |
| U73122 | 0.549 | 0.000 | −0.865 | PLC inhibitor |
| Rottlerin | 0.549 | 0.030 | −0.865 | inhibitor of p38 activated |
| | | | | kinases |
| 1400W | 0.556 | 0.033 | −0.847 | iNOS inhibitor |
| FLUNARIZINE | 0.560 | 0.036 | −0.836 | Calcium channels |
| cyclosporin A | 0.561 | 0.047 | −0.834 | calcineurin inhibitor |
| Shikonin | 0.576 | 0.058 | −0.795 | apoptosis inducer, p53 |
| | | | | dependent |
| geldanamycin | 0.586 | 0.101 | −0.772 | HSP90 inhibitor |
| 3,4-dichloroisocoumarin | 0.586 | 0.029 | −0.772 | granzyme B inhibitor |
| Ala-Ala-Phe-CMK | 0.599 | 0.068 | −0.738 | tripeptidyl peptidase II |
| | | | | inhibitor |
| 1-OLEOYL-2-ACETYL- | 0.604 | 0.056 | −0.727 | |
| GLYCEROL | | | | |
| THAPSIGARGIN | 0.608 | 0.031 | −0.719 | Notch inhibitor |
| TPEN | 0.608 | 0.044 | −0.717 | cell permable heavy |
| | | | | metal chelator |
| 1-OLEOYL-2-ACETYL- | 0.609 | 0.063 | −0.716 | |
| GLYCEROL | | | | |
| Betulinic acid | 0.612 | 0.039 | −0.708 | induces mitochondrial |
| | | | | permeability pore |
| | | | | opening |
| calphostin C | 0.613 | 0.054 | −0.707 | PKC inhibitor |
| Nigericin | 0.614 | 0.034 | −0.703 | induces intracellular |
| | | | | acidification |
| dibutyrylcyclic AMP | 0.615 | 0.081 | −0.701 | PKA activator |
| FK-506 | 0.616 | 0.002 | −0.699 | FKBP ligand |
| Boc-GVV-CHO | 0.617 | 0.082 | −0.696 | gamma secretase |
| | | | | inhibitor |
| Z-prolyl-prolinal | 0.619 | 0.063 | −0.692 | prolyl endopeptidase |
| | | | | inhibitor |
| Bestatin | 0.621 | 0.075 | −0.688 | aminopeptidase inhibitor |
| Fumonisin B1 | 0.622 | 0.058 | −0.685 | inhibits ceramide |
| | | | | synthase |
| cytochalasin B | 0.625 | 0.056 | −0.677 | F actin capper |
| Thiorphan | 0.627 | 0.052 | −0.673 | neutral endopeptidase |
| | | | | inhibitor |
| ACETYL-FARNESYL- | 0.631 | 0.044 | −0.664 | |
| CYSTEINE | | | | |
| 8-EPI-PROSTAGLANDIN F2a | 0.634 | 0.035 | −0.657 | |
| AG1478 | 0.635 | 0.117 | −0.655 | receptor tyr kinase |
| | | | | inhibitor. Broad spectrum |
| ANANDAMIDE (20:3, n-6) | 0.636 | 0.056 | −0.653 | |
| Wiskostatin | 0.639 | 0.000 | −0.646 | N-WASP inhibitor |
| Deprenyl | 0.641 | 0.046 | −0.642 | inhibits glyceraldehyde- |
| | | | | 3-phosphate |
| | | | | dehydrogenase |
| Huperzine A [(−)-Huperzine A] | 0.641 | 0.000 | −0.641 | acetylcholinesterase |
| | | | | inhibitor |
| AG213 (Tyrphostin 47) | 0.648 | 0.065 | −0.625 | EGF-R tyrosine kinase |
| | | | | inhibitor |
| 8,9-EPOXYEICOSATRIENOIC | 0.649 | 0.054 | −0.625 | |
| ACID | | | | |
| MDL-28170 | 0.652 | 0.070 | −0.617 | calpain inhibitor |
| Milrinone | 0.653 | 0.121 | −0.615 | phosphodiesterase |
| | | | | (PDE3) inhibitor |
| Monastrol | 0.656 | 0.044 | −0.607 | Eg5 inhibitor |
| SQ-29548 | 0.657 | 0.100 | −0.606 | Thromboxane A2 |
| | | | | antagonist |
| roscovitine | 0.659 | 0.000 | −0.602 | CDK inhibitor |
| Phenoxybenzamine | 0.660 | 0.036 | −0.600 | calmodulin antagonist |
| CIMATEROL | 0.661 | 0.126 | −0.598 | adrenoceptor agonist |
| | | | | (beta) |
| zVAD-FMK | 0.661 | 0.019 | −0.597 | Caspase inhibitor (broad |
| | | | | spectrum) |
| Hoechst 33342 (cell permeable) | 0.663 | 0.000 | −0.592 | DNA minor groove |
| (BisBenzimide) | | | | binder |
| NS-398 | 0.663 | 0.050 | −0.592 | Cox-2 inhibitor |
| ionomycin | 0.664 | 0.028 | −0.591 | Ca++ ionophore |
| PINACIDIL | 0.664 | 0.049 | −0.591 | Potassium channels |
| HA14-1 | 0.664 | 0.164 | −0.590 | Bcl-2 ligand induces |
| | | | | apoptosis |
| N- | 0.664 | 0.048 | −0.590 | |
| ARACHIDONOYLGLYCINE | | | | |
| GW-9662 | 0.669 | 0.049 | −0.581 | PPARgamma antagonist |
| 9-CIS RETINOIC ACID | 0.670 | 0.087 | −0.578 | retinoid RXR agonist |
| Mycophenolic acid | 0.670 | 0.012 | −0.577 | Inosine-5′- |
| | | | | monophosphate |
| | | | | dehydrogenase inhibitor |
| diphenyleneiodonium Cl | 0.671 | 0.095 | −0.576 | flavoprotein inhibitor |
| U-37883A | 0.673 | 0.045 | −0.572 | Potassium channels |
| 12(S)-HPETE | 0.673 | 0.034 | −0.571 | |
| Tanshinone IIA | 0.674 | 0.065 | −0.570 | AP-1 inhibitor |
| S-FARNESYL-L-CYSTEINE | 0.677 | 0.105 | −0.563 | |
| ME | | | | |
| PROSTAGLANDIN E1 | 0.678 | 0.116 | −0.561 | |
| AG-370 | 0.679 | 0.031 | −0.559 | PDGF receptor kinase |
| | | | | inhibitor |
| NapSul-Ile-Trp-CHO | 0.681 | 0.101 | −0.554 | Cathepsin L inhibitor |
| GRAYANOTOXIN III | 0.682 | 0.054 | −0.553 | Sodium channels |
| resveratrol | 0.682 | 0.083 | −0.551 | SIRT1 activator |
| indomethacin | 0.683 | 0.044 | −0.549 | cyclooxygenase inhibitor |
| 1-OCTADECYL-2- | 0.689 | 0.064 | −0.538 | |
| METHYLGLYCERO-3 PC | | | | |
| etoposide | 0.691 | 0.127 | −0.532 | topoisomerase II inhibitor |
| 1-STEAROYL-2- | 0.692 | 0.095 | −0.532 | |
| ARACHIDONOYL- | | | | |
| GLYCEROL | | | | |
| nocodazole | 0.692 | 0.029 | −0.531 | tubulin inhibitor |
| PROSTAGLANDIN J2 | 0.695 | 0.065 | −0.525 | |
| 5(S)-HPETE | 0.696 | 0.050 | −0.524 | |
| PROSTAGLANDIN A1 | 0.696 | 0.054 | −0.522 | |
| doxorubicin | 0.697 | 0.141 | −0.520 | topoisomerase II |
| | | | | inbhibitor, induces |
| | | | | apoptosis |
| KN-62 | 0.701 | 0.123 | −0.513 | CaM kinase II inhibitor |
| ZL3VS | 0.701 | 0.000 | −0.513 | proteasome inhibitor |
| Mevinolin (lovastatin) | 0.701 | 0.025 | −0.512 | Inhibitor HMG-CoA |
| | | | | reductase |
| LYSOPHOSPHATIDIC ACID | 0.703 | 0.133 | −0.509 | LPA receptor antagonist |
| AGC | 0.703 | 0.065 | −0.508 | |
| monensin | 0.708 | 0.018 | −0.499 | Na+ ionophore |
| W7 | 0.708 | 0.000 | −0.498 | calmodulin antagonist |
| C16 CERAMIDE | 0.709 | 0.067 | −0.496 | |
| NSC-95397 | 0.709 | 0.079 | −0.495 | CDC25 phosphatase |
| | | | | inhibitor |
| NS-1619 | 0.709 | 0.017 | −0.495 | Potassium channels |
| TOLBUTAMIDE | 0.710 | 0.019 | −0.494 | Potassium channels |
| C2 CERAMIDE | 0.711 | 0.069 | −0.493 | |
| RK-682 | 0.711 | 0.011 | −0.492 | VHR phosphatase |
| | | | | inhibitor |
| Pregnenolone 16alpha | 0.711 | 0.048 | −0.492 | PXR/SXR agonist |
| carbonitrile | | | | |
| pepstatin | 0.711 | 0.000 | −0.491 | protease inhibitor |
| Manoalide | 0.712 | 0.057 | −0.490 | Phospholipase A2 |
| | | | | inhibitor |
| N9-Isopropylolomoucine | 0.712 | 0.072 | −0.490 | CDC-2 kinase inhibitor |
| N,N- | 0.713 | 0.055 | −0.487 | sphingosine kinase |
| DIMETHYLSPHINGOSINE | | | | inhibitor |
| wortmannin | 0.714 | 0.138 | −0.486 | PI3Kinase, other kinases |
| | | | | inhibitor |
| PALMITYLETHANOLAMIDE | 0.714 | 0.042 | −0.486 | Cannabinoid CB2 agonist |
| RG-14620 | 0.715 | 0.016 | −0.483 | EGF-R tyrosine kinase |
| | | | | inhibitor |
| Arvanil | 0.716 | 0.043 | −0.482 | Vaniloid receptor agonist |
| damnacanthal | 0.716 | 0.117 | −0.482 | p56lck inhibitor |
| Gliotoxin | 0.718 | 0.114 | −0.478 | Inhibitor of 20S- |
| | | | | proteasome chymotrypsin |
| | | | | activity |
| YC-1 | 0.719 | 0.029 | −0.476 | guanylyl cyclase |
| | | | | stimulator |
| Cerulenin | 0.720 | 0.040 | −0.474 | Fatty acid biosynthesis |
| | | | | inhibitor |
| SB-415286 | 0.721 | 0.085 | −0.472 | GSK3beta inhibitor |
| BAY 11-7082 | 0.722 | 0.026 | −0.470 | IKK kinase inhibitor |
| C2 DIHYDROCERAMIDE | 0.724 | 0.079 | −0.466 | |
| LY-83583 | 0.725 | 0.119 | −0.465 | Inhibits NO-activation of |
| | | | | guanylate cyclase |
| U-74389G | 0.726 | 0.040 | −0.462 | superoxide/free-radical |
| | | | | inhibitor |
| ML9 | 0.728 | 0.015 | −0.458 | kinase inhibitor |
| epibatidine (+/−) | 0.729 | 0.180 | −0.457 | nicotinic cholinergic |
| | | | | agonist |
| 1-STEAROYL-2- | 0.729 | 0.065 | −0.456 | |
| LINOLEOYL-GLYCEROL | | | | |
| Xestospongin C | 0.729 | 0.106 | −0.456 | IP3 receptor blocker |
| PAF C18 | 0.729 | 0.104 | −0.455 | PAF receptor agonist |
| 9,10-OCTADECENOAMIDE | 0.730 | 0.054 | −0.454 | |
| PROSTAGLANDIN F2a | 0.732 | 0.033 | −0.450 | |
| forskolin | 0.732 | 0.034 | −0.449 | Adenylate cyclase |
| | | | | activator |
| L-NAME | 0.734 | 0.021 | −0.446 | NO synthesis inhibitor |
| AA-861 | 0.735 | 0.029 | −0.444 | 5-lipoxygenase inhibitor |
| Decylubiquinone | 0.736 | 0.027 | −0.442 | inhibits mitochondrial |
| | | | | permeability pore |
| | | | | opening |
| quercetin | 0.737 | 0.048 | −0.440 | kinase inhibitor plus other |
| LEUKOTRIENE E4 | 0.737 | 0.072 | −0.439 | |
| NPPB | 0.738 | 0.031 | −0.439 | Misc. channels |
| ARACHIDONOYL-PAF | 0.738 | 0.033 | −0.439 | |
| E-64-d | 0.738 | 0.094 | −0.438 | calpain/cathepsin |
| | | | | inhibitor |
| 6-KETO-PROSTAGLANDIN | 0.740 | 0.153 | −0.435 | |
| F1a | | | | |
| Amino-1,8-naphthalimide [4- | 0.741 | 0.138 | −0.433 | PARP inhibitor |
| Amino-1,8-naphthalimide] | | | | |
| NICARDIPINE | 0.741 | 0.047 | −0.433 | Calcium channels |
| 17-Allylamino-geldanamycin | 0.741 | 0.078 | −0.433 | HSP-90 inhibitor |
| dipyridamole | 0.742 | 0.011 | −0.431 | cGMP phosphodiesterase |
| | | | | inhibitor |
| Ac-Leu-Leu-Nle-CHO | 0.742 | 0.081 | −0.431 | calpain inhibitor |
| C8 CERAMIDE | 0.744 | 0.057 | −0.427 | |
| PIMOZIDE | 0.745 | 0.112 | −0.425 | Calcium channels |
| C8 DIHYDROCERAMIDE | 0.746 | 0.121 | −0.423 | |
| brefeldin A | 0.746 | 0.081 | −0.422 | ARF GEF inhibitor |
| PAF C18:1 | 0.747 | 0.075 | −0.420 | PAF receptor agonist |
| LFM-A13 | 0.749 | 0.107 | −0.417 | BTK inhibitor |
| NIFEDIPINE | 0.749 | 0.057 | −0.417 | Calcium channels |
| GLIPIZIDE | 0.749 | 0.056 | −0.417 | Potassium channels |
| MINOXIDIL SULFATE | 0.749 | 0.033 | −0.417 | Potassium channels |
| Rolipram | 0.750 | 0.111 | −0.414 | phosphodiesterase (PDE4) |
| | | | | inhibitor |
| PROSTAGLANDIN D2 | 0.751 | 0.083 | −0.413 | |
| PROSTAGLANDIN B1 | 0.753 | 0.056 | −0.408 | |
| valinomycin | 0.754 | 0.072 | −0.408 | K+ ionophore |
| olomoucine | 0.755 | 0.048 | −0.406 | CDK inhibitor |
| A-3 | 0.757 | 0.078 | −0.403 | kinase inhibitor |
| NITRENDIPINE | 0.758 | 0.058 | −0.400 | Calcium channels |
| 9(S)-HODE | 0.759 | 0.043 | −0.398 | |
| Phenanthridinone [6(5H)- | 0.766 | 0.000 | −0.384 | PARP inhibitor |
| Phenanthridinone] | | | | |
| 6,7-ADTN HBr | 0.767 | 0.147 | −0.383 | Dopamine agonist |
| Y-27632 | 0.768 | 0.000 | −0.380 | ROCK inhibitor |
| 1-HEXADECYL-2- | 0.769 | 0.041 | −0.379 | |
| ARACHIDONOYL- | | | | |
| GLYCEROL | | | | |
| siguazodan | 0.771 | 0.080 | −0.376 | phosphodiesterase |
| | | | | (PDE3) inhibitor |
| HA-1004 | 0.773 | 0.040 | −0.372 | kinase inhibitor |
| GW-5074 | 0.774 | 0.048 | −0.369 | cRAF1 kinase inhibitor |
| TOLAZAMIDE | 0.776 | 0.040 | −0.365 | Potassium channels |
| ADRENIC ACID (22:4, n-6) | 0.777 | 0.071 | −0.364 | |
| 24,25-DIHYDROXYVITAMIN | 0.777 | 0.081 | −0.363 | |
| D3 | | | | |
| CDC | 0.779 | 0.002 | −0.360 | 12-Lipoxygenase |
| | | | | inhibitor |
| 17-OCTADECYNOIC ACID | 0.780 | 0.030 | −0.358 | |
| E6 berbamine | 0.780 | 0.035 | −0.358 | calmodulin inhibitor |
| 5-HYDROXYDECANOATE | 0.781 | 0.052 | −0.357 | Potassium channels |
| Kavain (+/−) | 0.781 | 0.096 | −0.357 | voltage-dependent Na |
| | | | | channel inhibitor |
| N-PHENYLANTHRANILIC | 0.782 | 0.116 | −0.355 | Misc. channels |
| (CL) | | | | |
| L-744,832 | 0.783 | 0.019 | −0.354 | Ras farnesyltransferase |
| | | | | inhibitor |
| castanospermine | 0.784 | 0.051 | −0.351 | glucosidase inhibitor |
| QUINIDINE | 0.784 | 0.116 | −0.351 | Sodium channels |
| LOPERAMIDE | 0.786 | 0.088 | −0.347 | Calcium channels |
| PROCAINAMIDE | 0.787 | 0.179 | −0.346 | Sodium channels |
| 5(S)-HETE | 0.788 | 0.059 | −0.343 | |
| dibutyrylcyclic GMP | 0.789 | 0.193 | −0.342 | PKA activator |
| AM 92016 | 0.789 | 0.027 | −0.341 | Potassium channels |
| ZM336372 | 0.791 | 0.019 | −0.338 | raf inhibitor |
| PD 98059 | 0.791 | 0.066 | −0.338 | MEK inhibitor |
| CinnGEL 2Me | 0.792 | 0.090 | −0.336 | PTP1B inhibitor |
| PROSTAGLANDIN B2 | 0.796 | 0.122 | −0.330 | |
| Tyrphostin-8 | 0.796 | 0.115 | −0.329 | Calcineurin inhibitor |
| METHOPRENE ACID | 0.796 | 0.083 | −0.328 | Retinoid RXR agonist |
| HA1077 | 0.798 | 0.042 | −0.326 | inhibitor of Rho- |
| | | | | dependent kinases |
| TMB-8 | 0.798 | 0.052 | −0.325 | Intracellular calcium |
| CAPE | 0.799 | 0.045 | −0.324 | Antioxidant/NFkappa B |
| | | | | inhibitor |
| phorbol 12 myristate 13 acetate | 0.799 | 0.003 | −0.323 | PKC activator |
| MEAD ETHANOLAMIDE | 0.800 | 0.094 | −0.322 | Cannabinoid receptor |
| | | | | agonist |
| PHENTOLAMINE | 0.800 | 0.059 | −0.321 | Potassium channels |
| 12-METHOXYDODECANOIC | 0.801 | 0.128 | −0.320 | |
| ACID | | | | |
| Alrestatin | 0.801 | 0.001 | −0.320 | aldose reductase inhibitor |
| TETRANDRINE | 0.802 | 0.054 | −0.318 | Calcium channels |
| N-ACETYL-LEUKOTRIENE | 0.802 | 0.065 | −0.318 | |
| E4 | | | | |
| 5-KETOEICOSATETRAENOIC | 0.803 | 0.026 | −0.316 | |
| ACID | | | | |
| SDZ-201106 | 0.804 | 0.091 | −0.315 | Sodium channels |
| FCCP | 0.805 | 0.000 | −0.313 | mitochondrial uncoupler |
| EHNA HCl | 0.805 | 0.090 | −0.312 | Phosphodiesterase |
| | | | | (PDE2) |
| | | | | inhibitor/adenosine |
| | | | | deaminase inhibitor |
| AM-580 | 0.806 | 0.091 | −0.311 | |
| 15(S)-HETE | 0.807 | 0.096 | −0.309 | |
| WIN 55,212-2 | 0.810 | 0.052 | −0.305 | cannabinoid CB1/CB2 |
| | | | | agonist |
| C8 CERAMINE | 0.811 | 0.052 | −0.302 | |
| mastoparan | 0.812 | 0.159 | −0.301 | activates heterotrimeriuc |
| | | | | GTPases |
| NIGULDIPINE | 0.812 | 0.071 | −0.301 | Calcium channels |
| PHENYTOIN | 0.812 | 0.079 | −0.300 | Sodum channels |
| U-50488 | 0.813 | 0.052 | −0.298 | Calcium channels |
| decoyinine | 0.813 | 0.077 | −0.298 | lowers GTP levels |
| IBMX | 0.814 | 0.067 | −0.297 | PDE inhibitor (broad |
| | | | | spec), adenosineR agonist |
| Lavendustin A | 0.814 | 0.087 | −0.297 | tyr kinase inhibitor EGF- |
| | | | | R) |
| Ikarugamyin | 0.814 | 0.084 | −0.296 | inhibits clathrin coated pit |
| | | | | mediated endocytosis |
| PROSTAGLANDIN A2 | 0.815 | 0.204 | −0.295 | |
| FARNESYLTHIOACETIC | 0.815 | 0.143 | −0.295 | |
| ACID | | | | |
| 9a,11b-PROSTAGLANDIN F2 | 0.815 | 0.067 | −0.294 | |
| 9(S)-HPODE | 0.816 | 0.144 | −0.294 | |
| 2,5-ditertbutylhydroquinone | 0.816 | 0.076 | −0.293 | ER Ca++ ATPase |
| | | | | inhibitor |
| MINOXIDIL | 0.817 | 0.102 | −0.292 | Potassium channels |
| PHOSPHATIDIC ACID, | 0.819 | 0.039 | −0.288 | |
| DIPALMITOYL | | | | |
| Go6976 | 0.819 | 0.036 | −0.287 | PKC inhibitor |
| N-LINOLEOYLGLYCINE | 0.821 | 0.095 | −0.285 | |
| CARBACYCLIN | 0.822 | 0.018 | −0.283 | PPAR delta agonist |
| REV-5901 | 0.822 | 0.112 | −0.282 | 5-Lipoxygenase inhibitor |
| aphidicolin | 0.823 | 0.054 | −0.281 | DNA polymerase |
| | | | | inhibitor |
| Furoxan | 0.823 | 0.037 | −0.280 | NO donor |
| 1-ACYL-PAF | 0.823 | 0.126 | −0.280 | |
| RHC-80267 | 0.824 | 0.052 | −0.279 | DAG lipase inhibitor |
| MEAD ACID (20:3 n-9) | 0.824 | 0.167 | −0.279 | |
| Trequinsin | 0.824 | 0.098 | −0.279 | phosphodiesterase |
| | | | | (PDE3) inhibitor |
| ACONITINE | 0.825 | 0.059 | −0.278 | Sodium channels |
| juglone | 0.825 | 0.051 | −0.277 | PIN1 inhibitor(?) |
| cytochalasin D | 0.826 | 0.080 | −0.276 | F actin capper |
| genistein | 0.826 | 0.082 | −0.275 | tyrosine kinase inhibitor |
| GINGEROL | 0.827 | 0.029 | −0.273 | Intracellular calcium |
| 6-FORMYLINDOLO [3,2-B] | 0.828 | 0.080 | −0.273 | AHR agonist |
| CARBAZOLE | | | | |
| AG-490 | 0.828 | 0.067 | −0.272 | JAK2 inhibitor |
| SQ22536 | 0.829 | 0.025 | −0.270 | adenylate cyclase |
| | | | | inhibitor |
| 15- | 0.832 | 0.118 | −0.266 | |
| KETOEICOSATETRAENOIC | | | | |
| ACID | | | | |
| MAPP, D-erythro | 0.832 | 0.069 | −0.266 | |
| Nimesulide | 0.833 | 0.119 | −0.264 | Cox 2 inhibitor |
| DIAZOXIDE | 0.837 | 0.100 | −0.257 | Potassium channels |
| VERAPAMIL | 0.838 | 0.065 | −0.255 | Calcium channels |
| 17-PHENYL-TRINOR-PGE2 | 0.839 | 0.098 | −0.253 | |
| FLECAINIDE | 0.839 | 0.072 | −0.253 | Sodium channels |
| thalidomide | 0.839 | 0.033 | −0.252 | TNFalpha synthesis |
| | | | | inhibitor |
| AMANTIDINE | 0.840 | 0.013 | −0.252 | Misc. channels |
| DOCOSAHEXAENOIC ACID | 0.842 | 0.080 | −0.248 | |
| (22:6 n-3) | | | | |
| DRB | 0.843 | 0.087 | −0.246 | CKII inhibitor |
| KT-5720 | 0.843 | 0.069 | −0.246 | PKA inhibitor |
| dexamethasone | 0.844 | 0.118 | −0.245 | corticosteroid |
| MnTBAP | 0.844 | 0.078 | −0.245 | SOD mimetic |
| PROSTAGLANDIN I2 Na | 0.844 | 0.087 | −0.244 | |
| D609 | 0.844 | 0.057 | −0.244 | PC-PLC inhibitor |
| 24(S)-hydroxycholesterol | 0.844 | 0.055 | −0.244 | LXR agonist |
| 4- | 0.845 | 0.137 | −0.244 | |
| HYDROXYPHENYLRETINAMIDE | | | | |
| Ro 20-1724 | 0.846 | 0.059 | −0.242 | phosphodiesterase (PDE4) |
| | | | | inhibitor |
| BAPTA-AM | 0.846 | 0.121 | −0.242 | cell permeable Ca++ |
| | | | | chelator |
| Splitomycin | 0.846 | 0.082 | −0.241 | sir2p inhibitor |
| Hinokitiol | 0.847 | 0.070 | −0.240 | Iron chelator |
| TETRAHYDROCANNABINOL- | 0.847 | 0.092 | −0.240 | PPARgamma agonist |
| 7-OIC ACID | | | | |
| GF-109203X | 0.848 | 0.054 | −0.238 | PKC inhibitor |
| NIMODIPINE | 0.848 | 0.054 | −0.238 | Calcium channels |
| calpeptin | 0.849 | 0.071 | −0.235 | calpain inhibitor |
| bafilomycin A1 | 0.850 | 0.107 | −0.234 | vaculolar ATPase |
| | | | | inhibitor |
| swainsonine | 0.851 | 0.019 | −0.233 | protein glycosylation |
| | | | | inhibitor |
| manumycin A | 0.851 | 0.118 | −0.233 | ras farnesylation inhibitor |
| HBDDE | 0.851 | 0.087 | −0.233 | PKC inhibitor |
| Tosyl-Phe-CMK (TPCK) | 0.851 | 0.145 | −0.233 | serine protease inhibitor |
| PROSTAGLANDIN E2 | 0.857 | 0.042 | −0.223 | |
| SB 202190 | 0.857 | 0.000 | −0.222 | MAP kinase inhibitor |
| Cyclo [Arg-Gly-Asp-D-Phe-Val] | 0.858 | 0.147 | −0.221 | integrin inhibitor |
| 1-HEXADECYL-2-O- | 0.859 | 0.289 | −0.219 | |
| METHYL-GLYCEROL | | | | |
| 12(R)-HETE | 0.859 | 0.041 | −0.219 | |
| GLYBURIDE | 0.861 | 0.175 | −0.217 | Potassium channels |
| RWJ-60475-(AM)3 | 0.861 | 0.058 | −0.217 | CD45 phosphatase |
| | | | | inhibitor |
| LEUKOTOXIN A (9,10-EODE) | 0.861 | 0.037 | −0.216 | |
| H9 | 0.862 | 0.073 | −0.215 | kinase inhibitor |
| FLUFENAMIC ACID | 0.862 | 0.030 | −0.214 | Potassium channels |
| PROPAFENONE | 0.862 | 0.067 | −0.214 | Potassium channels |
| Bromo-7-nitroindazole [3- | 0.862 | 0.047 | −0.214 | NO synthase inhbitor |
| Bromo-7-nitroindazole] | | | | |
| bezafibrate | 0.864 | 0.066 | −0.211 | PPARalpha agonist |
| PROSTAGLANDIN F1a | 0.866 | 0.084 | −0.208 | |
| trifluoperazine | 0.867 | 0.100 | −0.206 | calmodulin inhibitor- |
| | | | | possibly only at high |
| | | | | concentrations! |
| DILTIAZEM | 0.868 | 0.082 | −0.205 | Calcium channels |
| LY-294002 | 0.868 | 0.047 | −0.204 | PI-3-Kinase inhibitor |
| BML-190 | 0.869 | 0.071 | −0.202 | |
| QUININE | 0.870 | 0.062 | −0.201 | Potassium channels |
| NIFLUMIC ACID | 0.870 | 0.050 | −0.201 | Misc. channels |
| MCI-186 | 0.871 | 0.083 | −0.200 | antioxidant, |
| | | | | cytoprotectant |
| CGP-37157 | 0.873 | 0.204 | −0.195 | inhibitor of mitochondrial |
| | | | | Na+Ca+2 exchange |
| E-4031 | 0.874 | 0.065 | −0.195 | Potassium channels |
| FLUSPIRILINE | 0.875 | 0.095 | −0.192 | Potassium channels |
| PAF C16 | 0.876 | 0.153 | −0.190 | PAF receptor agonist |
| PCO-400 | 0.882 | 0.070 | −0.182 | Potassium channels |
| tyrphostin 1 | 0.882 | 0.054 | −0.181 | Calcineurin inhibitor |
| PCA 4248 | 0.883 | 0.000 | −0.180 | PAF antagonist |
| Thiocitrulline [L-Thiocitrulline | 0.889 | 0.070 | −0.170 | bNOS inhibitor |
| HCl] | | | | |
| OBAA | 0.890 | 0.113 | −0.169 | phospholipase A2 |
| | | | | inhibitor |
| Ebselen | 0.890 | 0.095 | −0.168 | glutathione peroxidase |
| | | | | mimetic |
| FPL-64176 | 0.891 | 0.061 | −0.166 | Calcium channels |
| 5,6-EPOXYEICOSATRIENOIC | 0.892 | 0.076 | −0.164 | |
| ACID | | | | |
| CITCO | 0.894 | 0.184 | −0.162 | Const. androstane |
| | | | | receptor agonist |
| RETINOIC ACID, ALL TRANS | 0.894 | 0.056 | −0.162 | Retinoid RAR agonist |
| DOCOSAPENTAENOIC ACID | 0.895 | 0.064 | −0.161 | |
| ESTRADIOL | 0.895 | 0.045 | −0.160 | estrogen |
| Aminobenzamide (3-ABA) [3- | 0.895 | 0.048 | −0.160 | ADP ribose polymerase, |
| aminobenzamide (3-ABA)] | | | | apoptosis inhibitor |
| TRIM | 0.895 | 0.070 | −0.160 | bNOS/iNOS inhibitor |
| HISTAMINE | 0.896 | 0.131 | −0.158 | Histamine receptor |
| | | | | agonist |
| deoxynorjrimycin(1) | 0.899 | 0.144 | −0.154 | glucosidase inhibitor |
| YS035 | 0.900 | 0.202 | −0.152 | Calcium channels |
| Lycorine | 0.900 | 0.065 | −0.152 | inhibits TNFalpha |
| | | | | production |
| EICOSA-5,8-DIENOIC ACID | 0.902 | 0.140 | −0.149 | |
| (20:2 n-12) | | | | |
| anisomycin | 0.902 | 0.117 | −0.148 | MAP kinase activator |
| chelerythrine | 0.903 | 0.095 | −0.147 | PKC inhibitor |
| GAMMA-LINOLENIC ACID | 0.904 | 0.046 | −0.145 | |
| (18:3 n-6) | | | | |
| cyclopamine | 0.905 | 0.068 | −0.144 | Hedgehog pathway |
| | | | | inhibitor |
| 13(S)-HODE | 0.907 | 0.089 | −0.141 | |
| MISOPROSTOL, FREE ACID | 0.909 | 0.138 | −0.137 | Prostaglandin E receptor |
| | | | | agonist |
| Parthenolide | 0.913 | 0.071 | −0.131 | IkappaB kinase inhibitor |
| clozapine | 0.916 | 0.143 | −0.126 | dopamine antagonist |
| deoxymannojirimycin(1) | 0.917 | 0.191 | −0.125 | mannosidase inhibitor |
| AG-1296 | 0.919 | 0.168 | −0.122 | c-kit, FGF and PDGF |
| | | | | kinase inhibitor |
| ZM226600 | 0.922 | 0.179 | −0.117 | Potassium channels |
| DICHLOROBENZAMIL | 0.923 | 0.014 | −0.116 | Calcium channels |
| CA-074-Me | 0.924 | 0.099 | −0.114 | Cathepsin B inhibitor |
| TTNPB | 0.924 | 0.056 | −0.114 | Retinoid RAR agonist |
| 25-HYDROXYVITAMIN D3 | 0.925 | 0.072 | −0.113 | |
| PRIMA-1 | 0.926 | 0.054 | −0.110 | p53 reactivator |
| H-89 | 0.927 | 0.059 | −0.110 | PKA inhibitor |
| trichostatin-A | 0.927 | 0.098 | −0.109 | histone deacetylase |
| | | | | inhibitor |
| H7 | 0.931 | 0.048 | −0.104 | kinase inhibitor |
| Indirubin | 0.931 | 0.118 | −0.103 | GSK-3beta inhibitor |
| bongkrekic acid | 0.932 | 0.036 | −0.102 | ANT inhibitor |
| ANANDAMIDE (18:2, n-6) | 0.932 | 0.050 | −0.101 | |
| SP-600125 | 0.933 | 0.083 | −0.100 | JNK inhibitor |
| DANTROLENE | 0.934 | 0.129 | −0.098 | Intracellular calcium |
| EICOSADIENOIC ACID (20:2 | 0.938 | 0.118 | −0.093 | |
| n-6) | | | | |
| LYSO-PAF C16 | 0.941 | 0.092 | −0.087 | |
| tyrphostin AG-126 | 0.943 | 0.094 | −0.085 | tyrosine kinase inhibitor |
| IB-MECA | 0.944 | 0.088 | −0.083 | Adenosine receptor |
| | | | | agonist |
| LEUKOTOXIN B (12,13- | 0.945 | 0.047 | −0.081 | |
| EODE) | | | | |
| 1,2-DIOCTANOYL-SN- | 0.946 | 0.066 | −0.080 | |
| GLYCEROL | | | | |
| PAXILLINE | 0.948 | 0.003 | −0.078 | Potassium channels |
| METHOXY VERAPAMIL | 0.954 | 0.042 | −0.069 | Calcium channels |
| ARACHIDONAMIDE | 0.954 | 0.165 | −0.069 | |
| U-46619 | 0.957 | 0.074 | −0.063 | Thromboxane receptor |
| | | | | agonist |
| zaprinast | 0.962 | 0.072 | −0.055 | phosphodiesterase |
| | | | | (PDE1) inhibitor |
| DOCOSATRIENOIC ACID | 0.963 | 0.145 | −0.055 | |
| (22:3 n-3) | | | | |
| BEPRIDIL | 0.963 | 0.030 | −0.054 | Calcium channels |
| 11,12- | 0.965 | 0.013 | −0.052 | |
| EPOXYEICOSATRIENOIC | | | | |
| ACID | | | | |
| ENANTIO-PAF C16 | 0.965 | 0.016 | −0.051 | |
| HNMPA-(AM)3 | 0.966 | 0.118 | −0.049 | Insulin receptor TK |
| | | | | inhibitor |
| bumetanide | 0.967 | 0.127 | −0.048 | Na+K+Cl− cotransport |
| | | | | inhibitor |
| Bromo-cAMP [8-Bromo-cAMP] | 0.969 | 0.163 | −0.045 | PKA Activator |
| ARACHIDONIC ACID (20:4, n- | 0.969 | 0.046 | −0.045 | |
| 6) | | | | |
| tyrphostin AG-825 | 0.972 | 0.016 | −0.042 | HER-1,2 tyrosine kinase |
| | | | | inhibitor |
| AMILORIDE | 0.972 | 0.111 | −0.041 | Calcium channels |
| U-75302 | 0.972 | 0.082 | −0.041 | Leukotriene B4 receptor |
| | | | | antagonist |
| capsacin(E) | 0.973 | 0.241 | −0.040 | vanilloid receptor agonist |
| Leupeptin | 0.974 | 0.276 | −0.038 | protease inhibitor |
| LEUKOTRIENE B4 | 0.975 | 0.061 | −0.037 | |
| MY-5445 | 0.975 | 0.092 | −0.037 | phosphodiesterase |
| | | | | (PDE5) inhibitor |
| acetyl (N)-s-farnesyl-1-cysteine | 0.975 | 0.136 | −0.036 | farnesylation inhibitor |
| ascomycin (FK-520) | 0.976 | 0.098 | −0.035 | binds to FKBP inhibits |
| | | | | calcineurin |
| capsazepine | 0.979 | 0.052 | −0.030 | vanilloid receptor |
| | | | | antagonist |
| SPHINGOSINE | 0.980 | 0.121 | −0.030 | PKC inhibitor |
| LINOLEAMIDE | 0.981 | 0.160 | −0.027 | |
| BAY K-8644 | 0.982 | 0.047 | −0.027 | Calcium channels |
| Indirubin-3′-monoxime | 0.983 | 0.000 | −0.025 | GSK-3beta inhibitor |
| 5′-N- | 0.983 | 0.140 | −0.024 | adenosine receptor |
| Ethylcarboxamidoadenosine | | | | agonist |
| (NECA) | | | | |
| U-0126 | 0.984 | 0.002 | −0.023 | MEK inhibitor |
| D12-PROSTAGLANDIN J2 | 0.988 | 0.095 | −0.017 | PPARgamma agonist |
| cycloheximide-N-ethylethanoate | 0.990 | 0.121 | −0.014 | FKBP12 inhibitor |
| 16,16-DIMETHYL- | 0.991 | 0.187 | −0.013 | |
| PROSTAGLANDIN E2 | | | | |
| DIHYDROSPHINGOSINE | 1.000 | 0.198 | 0.000 | |
| AGGC | 1.001 | 0.054 | 0.001 | |
| L-NASPA | 1.002 | 0.160 | 0.003 | |
| 4-OXATETRADECANOIC | 1.003 | 0.244 | 0.004 | |
| ACID | | | | |
| 8-methoxymethyl-IBMX | 1.004 | 0.068 | 0.006 | phosphodiesterase |
| | | | | (PDE1) inhibitor |
| BENZAMIL | 1.004 | 0.008 | 0.006 | Calcium channels |
| ANANDAMIDE (22:4, n-6) | 1.005 | 0.090 | 0.007 | |
| prazocin | 1.008 | 0.159 | 0.011 | adrenoreceptor agonist |
| WY-14643 | 1.014 | 0.031 | 0.020 | PPAR alpha agonist |
| 7,7- | 1.014 | 0.171 | 0.021 | PLA2 inhibitor |
| DIMETHYLEICOSADIENOIC | | | | |
| ACID | | | | |
| Bromo-cGMP [8-Bromo-cGMP] | 1.015 | 0.205 | 0.021 | PKG Activator |
| pifithrin | 1.015 | 0.108 | 0.022 | p53 inhibitor |
| EICOSAPENTAENOIC ACID | 1.017 | 0.122 | 0.024 | |
| (20:5 n-3) | | | | |
| LINOLEIC ACID | 1.017 | 0.084 | 0.024 | |
| CIGLITAZONE | 1.027 | 0.182 | 0.038 | PPAR gamma agonist |
| LIPOXIN A4 | 1.033 | 0.093 | 0.046 | |
| LIDOCAINE | 1.035 | 0.105 | 0.049 | Sodium channels |
| SB 203580 | 1.036 | 0.163 | 0.051 | Suppressor of MAPKAP |
| | | | | kinase-2 |
| 2-FLUOROPALMITIC ACID | 1.036 | 0.098 | 0.051 | |
| ouabain | 1.037 | 0.019 | 0.053 | Na+K+ATPase inhibitor |
| ANANDAMIDE (20:4, n-6) | 1.038 | 0.061 | 0.054 | |
| 12(S)-HETE | 1.039 | 0.100 | 0.055 | |
| FLUPROSTENOL | 1.041 | 0.090 | 0.058 | |
| 1-HEXADECYL-2- | 1.042 | 0.161 | 0.060 | |
| METHYLGLYCERO-3 PC | | | | |
| IAA-94 | 1.043 | 0.010 | 0.061 | Misc. channels |
| yohimbine | 1.043 | 0.000 | 0.061 | adrenoreceptor antagonist |
| | | | | (alpha) |
| PP2 | 1.044 | 0.122 | 0.062 | Src family tyrosine kinase |
| | | | | inhibitor |
| 9b,11a-PROSTAGLANDIN F2 | 1.044 | 0.223 | 0.062 | |
| AM-251 | 1.046 | 0.105 | 0.065 | |
| SB-431542 | 1.048 | 0.090 | 0.068 | ALK4, ALK5, ALK7 |
| | | | | inhibitor |
| (R)-METHANANDAMIDE | 1.049 | 0.017 | 0.068 | Cannabinoid CB1 agonist |
| beta-lapachone | 1.050 | 0.091 | 0.070 | topoisomerase 1 inhibitor |
| CYCLOPIAZONIC ACID | 1.051 | 0.071 | 0.072 | Intracellular calcium |
| 13-CIS RETINOIC ACID | 1.053 | 0.069 | 0.074 | |
| DL-DIHYDROSPHINGOSINE | 1.054 | 0.129 | 0.076 | |
| DL-PDMP | 1.056 | 0.127 | 0.078 | |
| clonidine | 1.062 | 0.145 | 0.087 | adrenoreceptor agonist |
| | | | | (alpha) |
| 2-HYDROXYMYRISTIC ACID | 1.062 | 0.092 | 0.087 | |
| PHENAMIL | 1.063 | 0.062 | 0.088 | Sodium channels |
| 5,8,11-EICOSATRIYNOIC | 1.067 | 0.043 | 0.094 | |
| ACID | | | | |
| LINOLENIC ACID (18:3 n-3) | 1.069 | 0.280 | 0.096 | |
| MG-132 | 1.070 | 0.094 | 0.097 | proteasome inhibitor |
| propranolol (S—) | 1.070 | 0.000 | 0.098 | adrenoceptor antagonist |
| | | | | (beta) |
| propidium iodide | 1.072 | 0.147 | 0.100 | DNA intercalator |
| CLOPROSTENOL Na | 1.073 | 0.069 | 0.101 | prostaglandin FR receptor |
| | | | | agonist |
| AMIODARONE | 1.074 | 0.060 | 0.102 | Calcium channels |
| MAPP, L-erythro | 1.075 | 0.093 | 0.104 | |
| DL-PPMP | 1.078 | 0.110 | 0.108 | |
| Curcumin | 1.081 | 0.078 | 0.112 | NFkappaB inhibitor |
| LEUKOTRIENE C4 | 1.082 | 0.146 | 0.114 | |
| BW-B 70C | 1.084 | 0.073 | 0.116 | 5 lipoxygenase inhibitor |
| SU-4312 | 1.085 | 0.000 | 0.118 | VEGF-R (Flk-1) tyrosine |
| | | | | kinase |
| 2- | 1.086 | 0.074 | 0.119 | Cannabinoid CB1 agonist |
| ARACHIDONOYLGLYCEROL | | | | |
| C-PAF | 1.086 | 0.169 | 0.119 | PAF receptor agonist |
| CIRAZOLINE | 1.088 | 0.087 | 0.121 | adrenoreceptor agonist |
| | | | | (alpha) |
| SKF-96365 | 1.097 | 0.025 | 0.134 | Calcium channels |
| L-cis-DILTIAZEM | 1.100 | 0.054 | 0.137 | Calcium channels |
| zardaverine | 1.106 | 0.105 | 0.145 | phosphodiesterase |
| | | | | (PDE1/2) inhibitor |
| QX-314 | 1.107 | 0.099 | 0.146 | Sodium channels |
| piroxicam | 1.107 | 0.308 | 0.147 | COX1 inhibitor |
| Helenalin | 1.112 | 0.059 | 0.154 | NFkappaB inhibitor |
| Ro 31-8220 | 1.116 | 0.085 | 0.158 | PKC inhibitor |
| LY-171883 | 1.124 | 0.043 | 0.168 | |
| GM6001 | 1.128 | 0.066 | 0.173 | broad spectrum MMP |
| | | | | inhibitor |
| A-23187 | 1.130 | 0.048 | 0.176 | Calcium ionophore |
| ML7 | 1.143 | 0.058 | 0.192 | kinase inhibitor |
| MBCQ | 1.145 | 0.103 | 0.195 | phosphodiesterase |
| | | | | (PDE5) inhibitor |
| Blebbistatin | 1.146 | 0.127 | 0.196 | Myosin II inhibitor |
| EICOSATRIENOIC ACID (20:3 | 1.147 | 0.046 | 0.198 | |
| n-3) | | | | |
| CLOFIBRATE | 1.165 | 0.044 | 0.221 | |
| B581 | 1.171 | 0.094 | 0.228 | farnesyltransferase |
| | | | | inhibitor |
| LEUKOTRIENE D4 | 1.176 | 0.170 | 0.234 | |
| serotonin | 1.203 | 0.266 | 0.266 | serotonin receptor |
| vinpocetine | 1.214 | 0.131 | 0.280 | phosphodiesterase |
| | | | | (PDE1) inhibitor |
| puromycin | 1.215 | 0.000 | 0.281 | protein synthesis inhibitor |
| 15d-Prostaglandin J2 | 1.226 | 0.196 | 0.294 | |
| 13(S)-HPODE | 1.239 | 0.223 | 0.309 | |
| 1,2-DIDECANOYL- | 1.240 | 0.215 | 0.310 | |
| GLYCEROL (10:0) | | | | |
| tyrphostin AG-370 | 1.243 | 0.026 | 0.314 | PDGF-R tyrosine kinase |
| | | | | inhibitor |
| 13- | 1.248 | 0.147 | 0.320 | |
| KETOOCTADECADIENOIC | | | | |
| ACID | | | | |
| 14,15- | 1.250 | 0.074 | 0.322 | |
| EPOXYEICOSATRIENOIC | | | | |
| ACID | | | | |
| DIHOMO-GAMMA- | 1.251 | 0.147 | 0.323 | |
| LINOLENIC ACID | | | | |
| BADGE | 1.270 | 0.155 | 0.345 | PPARg antagonist |
| 15(S)-HPETE | 1.285 | 0.229 | 0.361 | |
| K252A | 1.287 | 0.100 | 0.364 | Kinase inhibitor (Broad |
| | | | | spectrum) |
| 1,2-DIOLEOYL-GLYCEROL | 1.359 | 0.137 | 0.443 | |
| (18:1) | | | | |
| DIINDOLYLMETHANE | 1.381 | 0.203 | 0.465 | AHR agonist |
| PP1 | 1.418 | 0.000 | 0.503 | src family trosine kinase |
| | | | | inhibitor |
| 1-HEXADECYL-2-O- | 1.432 | 0.155 | 0.518 | |
| ACETYL-GLYCEROL | | | | |
| 13,14-DIHYDRO-PGE1 | 1.502 | 0.179 | 0.587 | |
| tyrphostin 9 | 1.592 | 0.000 | 0.671 | PDGF-R tyrosine kinase |
| | | | | inhibitor |
| 1,25-DIHYDROXYVITAMIN | 1.599 | 0.252 | 0.677 | |
| D3 | | | | |
| AG-879 | 1.869 | 0.071 | 0.902 | NGF receptor inhibitor |
| Compound E | 2.696 | 0.621 | 1.431 | gamma-secretase |
| | | | | inhibitor |
| Lethals | | | | |
| 4-AMINOPYRIDINE | lethal | | | Potassium channels |
| FIPRONIL | lethal | | | Misc. channels |
| PENITREM A | lethal | | | Potassium channels |
| RYANODINE | lethal | | | Intracellular calcium |
| VERATRIDINE | lethal | | | Sodium channels |
| cantharidin | lethal | | | PP2A inhibitor |
| cypermethrin | lethal | | | calcineurin inhibitor |

TABLE 2
Screen of 88 FDA drugs in 96-well plates

| | Day 3 | Log2 | Day 6 | Avg |
|---|---|---|---|---|
| Drug Name | Fold | (fold) | Fold | (3 + 6) |
| Methotrexate | 0.107 | −3.231 | 0.081 | 0.094 |
| Dactinomycin | 0.119 | −3.068 | | 0.119 |
| Gemcitabine HCl | 0.158 | −2.666 | 0.051 | 0.104 |
| Vinblastine sulfate | 0.184 | −2.445 | 0.131 | 0.158 |
| Floxuridine | 0.217 | −2.204 | 0.080 | 0.149 |
| Vincristine sulfate | 0.233 | −2.100 | 0.394 | 0.314 |
| Bortezomib | 0.248 | −2.013 | 0.967 | 0.607 |
| Mitomycin C | 0.279 | −1.842 | 0.129 | 0.204 |
| Paclitaxel | 0.285 | −1.808 | 1.104 | 0.695 |
| Topotecan HCl | 0.330 | −1.600 | 0.137 | 0.233 |
| Triethylenemelamine | 0.349 | −1.519 | 0.253 | 0.301 |
| (TEM) | | | | |
| Thiotepa | 0.353 | −1.501 | 0.335 | 0.344 |
| Daunorubicin HCl | 0.358 | −1.484 | 0.096 | 0.227 |
| Rapamycin | 0.431 | −1.216 | 0.308 | 0.369 |
| Decitabine | 0.474 | −1.077 | 0.663 | 0.569 |
| Docetaxel | 0.541 | −0.887 | 0.487 | 0.514 |
| Carmustine | 0.565 | −0.823 | 0.426 | 0.496 |
| Pemetrexed | 0.581 | −0.783 | 0.149 | 0.365 |
| Vorinostat | 0.614 | −0.703 | 0.883 | 0.749 |
| Teniposide | 0.625 | −0.679 | 0.642 | 0.634 |
| Altretamine | 0.649 | −0.625 | 0.574 | 0.611 |
| Lomustine, CCNU | 0.651 | −0.619 | 0.773 | 0.712 |
| Nilotinib | 0.653 | −0.615 | 0.956 | 0.804 |
| Celecoxib | 0.676 | −0.565 | 0.834 | 0.755 |
| Imatinib | 0.679 | −0.558 | 0.670 | 0.674 |
| Fluorouracil | 0.733 | −0.448 | 0.806 | 0.770 |
| Dexrazoxone | 0.743 | −0.428 | 0.454 | 0.599 |
| Sorafenib | 0.752 | −0.410 | 1.339 | 1.046 |
| Mitoxantrone | 0.810 | −0.303 | 0.955 | 0.883 |
| Mitotane | 0.827 | −0.274 | 0.938 | 0.882 |
| Exemestane | 0.843 | −0.246 | 0.634 | 0.739 |
| Sunitinib | 0.845 | −0.243 | 0.788 | 0.816 |
| Fulvestrant | 0.858 | −0.221 | 1.486 | 1.172 |
| Acrichine | 0.863 | −0.212 | 1.229 | 1.046 |
| Irinotecan HCl | 0.870 | −0.201 | 0.541 | 0.705 |
| Valrubicin | 0.876 | −0.190 | 0.679 | 0.778 |
| Vinorelbine tartrate | 0.877 | −0.190 | 0.645 | 0.761 |
| Hydroxyurea | 0.880 | −0.184 | 1.099 | 0.990 |
| Gefitinib | 0.882 | −0.182 | 0.890 | 0.886 |
| Clofarabine | 0.893 | −0.164 | 0.843 | 0.868 |
| Etoposide | 0.911 | −0.134 | 0.722 | 0.817 |
| Lenalidomide | 0.922 | −0.117 | 0.784 | 0.853 |
| Zolendronic acid | 0.923 | −0.115 | 1.076 | 0.999 |
| Lapatinib | 0.935 | −0.097 | 1.017 | 0.976 |
| Nitrogen mustard | 0.939 | −0.090 | 0.683 | 0.811 |
| Doxorubicin HCl | 0.939 | −0.090 | 1.228 | 1.084 |
| Capecitabine | 0.947 | −0.078 | 1.087 | 1.017 |
| Arsenic trioxide | 0.950 | −0.074 | 1.012 | 0.981 |
| Anastrozole | 0.964 | −0.053 | 1.036 | 1.000 |
| Uracil mustard | 0.966 | −0.050 | 0.773 | 0.869 |
| Tamoxifen citrate | 0.968 | −0.046 | 0.869 | 0.919 |
| Estramustine disodium | 0.971 | −0.042 | 0.872 | 0.922 |
| phosphate | | | | |
| Tretinoin | 0.972 | −0.040 | 0.769 | 0.871 |
| Temozolomide | 0.978 | −0.032 | 1.298 | 1.138 |
| Ifosfamide | 0.982 | −0.026 | 1.026 | 1.004 |
| Cyclophosphamide | 0.982 | −0.025 | 0.732 | 0.857 |
| Aminolevulinic acid | 0.989 | −0.016 | 0.805 | 0.897 |
| Methoxsalen | 0.989 | −0.016 | 0.866 | 0.927 |
| Pipobroman | 0.992 | −0.011 | 0.714 | 0.853 |
| Dacarbazine | 0.993 | −0.010 | 0.927 | 0.960 |
| Ixabepilone | 0.994 | −0.009 | 0.536 | 0.765 |
| Melphalan | 1.001 | 0.001 | 0.876 | 0.939 |
| Erlotinib HCl | 1.030 | 0.042 | 0.942 | 0.986 |
| Streptozocin | 1.030 | 0.043 | 0.856 | 0.943 |
| Carboplatin | 1.042 | 0.059 | 1.276 | 1.159 |
| Bleomycin | 1.043 | 0.060 | 0.000 | 0.521 |
| Cladribine | 1.051 | 0.072 | 0.652 | 0.852 |
| Imiquimod | 1.061 | 0.086 | 0.715 | 0.888 |
| Cytarabine HCl | 1.072 | 0.100 | 1.153 | 1.112 |
| Mercaptopurine | 1.072 | 0.101 | 0.818 | 0.945 |
| Thalidomide | 1.099 | 0.136 | 0.918 | 1.008 |
| Cisplatin | 1.106 | 0.145 | 1.120 | 1.113 |
| Azacitidine | 1.115 | 0.157 | 0.976 | 1.045 |
| Nelarabine | 1.122 | 0.166 | 1.266 | 1.194 |
| Busulfan | 1.122 | 0.166 | 0.750 | 0.936 |
| Chlorambucil | 1.129 | 0.175 | 0.915 | 1.022 |
| Amifostine | 1.187 | 0.248 | 1.291 | 1.239 |
| Fludarabine | 1.192 | 0.253 | 1.191 | 1.191 |
| Oxaliplatin | 1.227 | 0.296 | 0.936 | 1.082 |
| Pentostatin | 1.232 | 0.301 | 0.669 | 0.951 |
| Plicamycin | 1.248 | 0.320 | 0.113 | 0.680 |
| Letrozole | 1.263 | 0.337 | 1.342 | 1.302 |
| Raloxifene HCl | 1.263 | 0.337 | 0.936 | 1.100 |
| Megestrol acetate | 1.336 | 0.418 | 1.101 | 1.219 |
| Procarbazine | 1.385 | 0.470 | 0.854 | 1.119 |
| Thioguanine | 1.409 | 0.495 | 1.375 | 1.392 |
| Dasatinib | 1.435 | 0.521 | 0.736 | 1.085 |
| Allopurinol | 2.430 | 1.281 | 1.565 | 1.997 |

TABLE 3
Summary of all hits from 569 compounds screened

| Drug Name | Fold | Log2(fold) | Screen | Proposed Role |
|---|---|---|---|---|
| Methotrexate | 0.107 | −3.231 | NCI | DHFR inhibitor |
| Dactinomycin | 0.119 | −3.068 | NCI | transcription |
| | | | | inhibitor |
| Gemcitabine HCl | 0.158 | −2.666 | NCI | nucleoside analog |
| methotrexate | 0.160 | −2.648 | ICCB | DHFR inhibitor |
| triptolide | 0.170 | −2.553 | ICCB | apoptosis inducer |
| Vinblastine sulfate | 0.184 | −2.445 | NCI | tubulin inhibitor |
| vinblastine | 0.204 | −2.290 | ICCB | tubulin inhibitor |
| Floxuridine | 0.217 | −2.204 | NCI | DNA synthesis |
| | | | | inhibitor |
| Vincristine sulfate | 0.233 | −2.100 | NCI | tubulin inhibitor |
| Bortezomib | 0.248 | −2.013 | NCI | proteasome inhibitor |
| tunicamycin | 0.263 | −1.925 | ICCB | glycosylation |
| | | | | inhibitor |
| paclitaxel | 0.274 | −1.870 | ICCB | microtubule |
| | | | | stabilizer |
| Mitomycin C | 0.279 | −1.842 | NCI | cross links DNA |
| Paclitaxel | 0.285 | −1.808 | NCI | microtubule |
| | | | | stabilizer |
| okadaic acid | 0.305 | −1.713 | ICCB | PP1 PP2A inhibitor |
| camptothecin | 0.326 | −1.619 | ICCB | topoisomerase 1 |
| | | | | inhibitor |
| Topotecan HCl | 0.330 | −1.600 | NCI | topoisomerase 1 |
| | | | | inhibitor |
| cycloheximide | 0.337 | −1.568 | ICCB | protein synthesis |
| | | | | inhibitor |
| tamoxifen | 0.339 | −1.562 | ICCB | estrogen antagonist |
| Triethylenemelamine | 0.349 | −1.519 | NCI | DNA synthesis |
| (TEM) | | | | inhibitor |
| Thiotepa | 0.353 | −1.501 | NCI | cross links DNA |
| Daunorubicin HCl | 0.358 | −1.484 | NCI | intercalates DNA |
| ICRF-193 | 0.364 | −1.459 | ICCB | topo II inhibitor that |
| | | | | does not cause DNA |
| | | | | breaks |
| calyculin A | 0.413 | −1.276 | ICCB | PP1, PP2a inhibitor |
| aristolochic acid | 0.422 | −1.243 | ICCB | phospholipase A2 |
| | | | | inhibitor |
| rapamycin | 0.429 | −1.220 | ICCB | FRAP inhibitor |
| Rapamycin | 0.431 | −1.216 | NCI | FRAP inhibitor |
| actinomycin D | 0.438 | −1.192 | ICCB | transcription |
| | | | | inhibitor |
| alamethicin | 0.445 | −1.167 | ICCB | monovalent cation |
| | | | | ionophor |
| oligomycin A | 0.457 | −1.131 | ICCB | Fo ATP synthase |
| | | | | inhibitor |
| mitomycin C | 0.470 | −1.088 | ICCB | cross links DNA |
| Latrunculin B | 0.478 | −1.064 | ICCB | actin inhibitor |
| Piceatannol | 0.493 | −1.020 | ICCB | Syk inhibitor |
| 10- | 0.525 | −0.930 | ICCB | topoisomerase 1 |
| hydroxycamptothecin | | | | inhibitor |
| 5-iodotubercidin | 0.530 | −0.917 | ICCB | ERK-2 inhibitor |
| Compound E | 2.696 | 1.431 | ICCB | gamma-secretase |
| | | | | inhibitor |

Raf-1^{gof} TSC flies were treated for three days with 5-iodotubercidin rapamycin, or Compound E (a Notch inhibitor). 5-iodotubercidin is a MEK/Dsor inhibitor that blocks MAPK signaling downstream of RAF, and rapamycin is a mTOR/TOR inhibitor that blocks several pathways that depend on TORC1 signaling (Zoncu et al., 2011) (FIG. 2E and FIG. 5A). Control flies were treated with DMSO. The effects of agent treatment were visualized with an esg-GFP reporter that marks tumor cells generally (green, upper panel in FIG. 5A) and with Delta staining, which specifically marks the TSCs (red, lower panel in FIG. 5A). 5-iodotubercidin and rapamycin reduced tumor growth while Compound E increased tumor growth. Thus, two compounds identified by the screening assay each inhibited a different pathway underlying the biology of TSCs and thus inhibited tumor growth, while another pathway-specific compound identified by the screening assay increased tumor growth.

To verify that the pathways were indeed required for the growth of Raf-1^{gof} tumors, RNAi was used to knockdown the targets of each inhibitor. That is, the requirement for MEK and TOR signaling was confirmed by RNAi against Dsor, and RNAi against Raptor, a required component of the rapamycin-sensitive TOR complex (called TORC1). The genetic effects were somewhat stronger than the drug effects in part because in the genetic experiments, RNAi constructs were co-expressed with the Raf-1^{gof} oncogene, whereas in the drug experiments, the Raf-1^{gof} transgene was activated for 24 hours prior to the drug treatments.

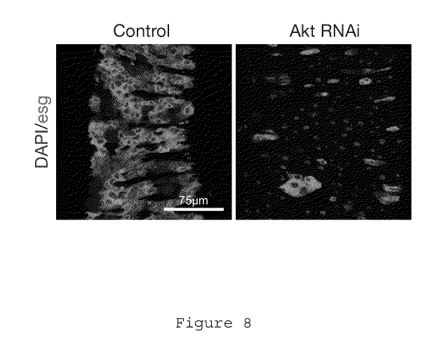

The effect of Compound E was confirmed by expressing a Notch Dominant-Negative transgene in Raf-1^{gof} TSCs: both resulted in a dramatic expansion of the tumor. Flies were crossed to a standard yv stock as a control. Thus, it was found that in each case the RNAi effects phenocopied the drug effects (FIG. 5B). Moreover, it was found that an upstream regulator of mTOR, AKT, was also required in Drosophila TSCs, further confirming the importance of TOR signaling in this model (FIG. 8). These results demonstrate that the Drosophila TSCs are sensitive not only to compounds that inhibit downstream effectors of the RAF-1^{gof} oncogene, but also to compounds that inhibit the parallel AKT/mTOR growth pathway. Drosophila TSCs thus mimic an additional feature of many mammalian cancers: the dual requirement for both RAS/MAPK and AKT/mTOR signaling (Downward, 2003).

The third pathway-specific inhibitor identified by the screen, Compound E, was distinct from the other hits in that it augmented tumor growth and luciferase activity rather than suppressing it (FIG. 2E and FIG. 5A). Compound E is a gamma-secretase inhibitor that blocks activation of the Notch pathway, a pathway that is required for ISC daughter cells to differentiate (Micchelli et al., 2006; Ohlstein et al., 2006). In the absence of Notch signaling, stem cell daughters continue to divide, forming stem cell-like tumors over the course of weeks. Strikingly, Notch inhibition in the context of the Raf-1^{gof} oncogene greatly accelerated growth of the tumor, which occupied the entire gut epithelium within days. This demonstrated that the RAF-1^{gof} model is a powerful way to identify Notch inhibitors, as it results in robust growth rather than cytotoxicity. Notch inhibitors are a highly sought-after class of compounds because they can inhibit the growth of several cancers, including colorectal cancers (Koch et al., 2010). Collectively, the identification of MAPK, mTOR, and Notch pathway inhibitors revealed aspects of the underlying biology of Raf-1^{gof} TSCs, and demonstrated that the model is sensitive to inhibitors of both stem cell maintenance and differentiation.

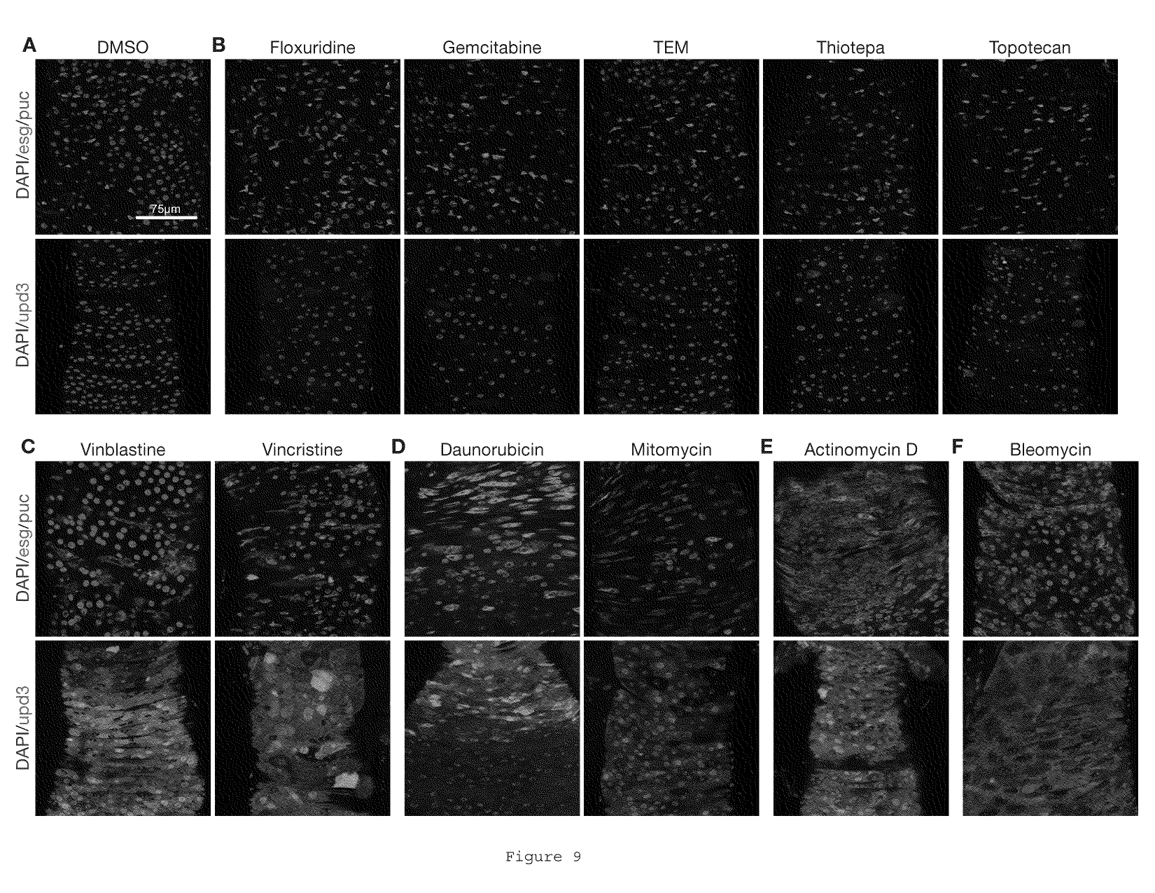

In addition to identifying pathway inhibitors, the screen also identified several compounds that were generally cytotoxic, including several FDA-approved anti-cancer drugs (FIG. 2E; Tables 1-3). These drugs had stronger inhibitory effects on tumor growth than the pathway-specific inhibitors, as revealed by both luciferase activity (FIG. 2E and FIG. 4) and visualization of the tumors (FIG. 4A and FIG. 9). Moreover, they eliminated virtually all of the Delta positive TSCs, consistent with the fact that FDA drugs are selective for rapidly dividing cells. Indeed, Drosophila stem cells that proliferated at a slower pace were not inhibited by these drugs (see below). The identification of FDA-approved anti-cancer drugs in the present screen underscores the relevance of using Drosophila in chemical screens: although Drosophila is highly diverged from mammals, the strong degree of molecular and cellular conservation between flies and mammals allows for the identification of small molecules in the transgenic fly that can be effective in humans.

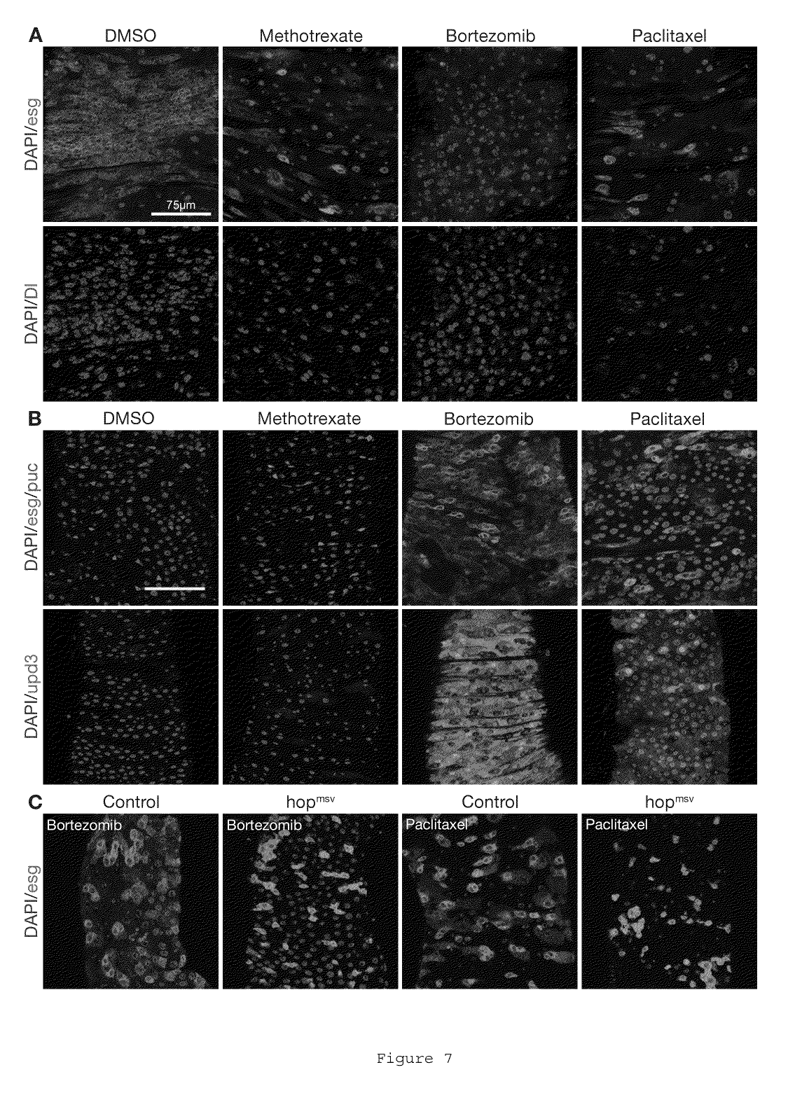

Since one of the benchmarks for anti-cancer drugs is that they should have greater effects on tumor cells than on wild-type cells, the effects of screen hits were tested on wild-type ISCs. Specifically, the three pathway-specific inhibitors identified by the screen and thirteen of the FDA-approved anti-cancer drug hits were tested. The effects of three representative FDA-approved drugs, methotrexate, bortezomib and paclitaxel, on Raf-1^{gof} tumors are shown in FIG. 7A. Flies were treated with DMSO alone as a control. All of the FDA drugs were potent inhibitors of Raf-1^{gof} tumors, as visualized by esg-GFP (green, upper panels in FIG. 7A) and by Delta staining of the same dissected guts (red, lower panels in FIG. 7A). The effect of each drug on wild-type guts was visualized with an esg-Gal4 reporter marking ISC/EBs progenitors (green in FIG. 7B) and a puc-LacZ (puckered-LacZ) reporter for JNK signaling (red in FIG. 7B) in upper panels, and with an unpaired3-GFP (upd3-GFP) reporter for JAK-STAT signaling in lower panels. Methotrexate, like the DMSO control, had no effect on the expression of any of the reporters, while bortezomib and paclitaxel affected both the microenvironment by inducing expression of puc-lacZ (red, upper panel) and upd3-GFP (green, lower panel) in EC cells, and by inducing an expansion of esg-expressing progenitor cells.

Thus, consistent with the expectations of anti-cancer drugs, each of the three pathway inhibitors, and 6 of the FDA drugs, had no discernable effects in wild-type ISCs (FIG. 7B and FIGS. 9A-B). All of the FDA drugs in this subset were S-phase inhibitors, exemplified by methotrexate (FIG. 7B). In contrast, the other 7 FDA-approved anti-cancer drugs had strong effects on wild-type ISCs. However, rather than killing the ISCs, they surprisingly promoted their proliferation. These drugs included the proteasome inhibitor bortezomib and the M-phase inhibitor paclitaxel (FIG. 7B), as well as several others (FIGS. 9C and 9E). This finding presents a paradox because each of these FDA drugs eliminated virtually all of the TSCs and yet promoted the proliferation of wild-type ISCs.

To understand how these compounds stimulated wild-type ISCs, the possibility that the compounds might affect the microenvironment was investigated, because stress in the EC daughter cells, a major component of the ISC microenvironment (Ohlstein et al., 2006), has been shown to induce ISC compensatory over-proliferation, i.e., transient overgrowth to replace damaged and dying daughter cells (Amcheslaysky et al., 2009; Jiang et al., 2009). For example, subjecting the fly intestine to the detergent dextran sulfate sodium (DSS) or to pathogen infection, induces ECs to activate the JNK stress-related pathway and to express IL-6 related cytokines (Unpaireds/Upds) (Jiang et al., 2009; Buchon et al., 2009; Apidianakis et al., 2009). The Upds activate JAK-STAT signaling in the ISCs, driving them to overproliferate. To determine if these same pathways are activated in response to FDA anti-cancer drugs, the puc-lacZ reporter was used to monitor JNK signaling, and a Gal-4 enhancer trap to monitor expression of Upd-3, the Upd cytokine preferentially expressed in the gut in response to injury (Jiang et al., 2009). A clear correlation was observed between the expression of these reporters and the effect of FDA drugs on wild-type cells: drugs like methotrexate that did not induce wild-type stem cell growth expressed neither reporter, whereas drugs that stimulated wild-type ISC proliferation, such as bortezomib and paclitaxel, induced expression of both the JNK puc-lacZ reporter and the Upd-3 reporter in the ECs (FIG. 7B and FIG. 9). Moreover, drug-induced ISC overproliferation was reduced in animals expressing a hypomorphic JAK allele, hop^{msv1} (FIG. 7C), demonstrating that JAK-STAT signaling is required for this response.

These results define a third class of inhibitors: compounds that both block TSCs and affect the microenvironment. All of the compounds that were identified in this class are FDA-approved anti-cancer drugs that either promote DNA damage or inhibit M-phase, transcription, or the proteasome (FIG. 7B and FIGS. 9C-E). Interestingly, additional drugs were found that affected the microenvironment but that could not block TSCs. For example, in experiments with bleomycin, a drug that induces DNA damage and that was previously shown to induce ISC overproliferation (Amcheslaysky et al., 2009), bleomycin was found to affect the microenvironment in the same way as the other drugs; it induced JNK and JAK-STAT signaling, yet it failed to block TSCs (FIGS. 4, 6F, and 9F). This finding demonstrates that stress effects on the microenvironment are not sufficient to arrest tumor growth, which suggests that the drugs that were identified have two separable activities: (1) the ability to block TSCs independent of the microenvironment, and (2) the ability to induce the microenvironment to express JNK and JAK-STAT pathways. This sets an important precedent for the interpretation of mammalian tissue culture screens, suggesting that drugs that can kill CSCs outside of the microenvironment may have counterproductive effects in vivo, where in the context of the microenvironment they may promote overproliferation and tumor initiation of the very cells that they are supposed to kill. Indeed, although microenvironment-induced cell proliferation is a normal tissue-repair response, it has been shown to induce tumorigenesis in Drosophila (Apidianakis et al., 2009) and mouse models (Bromberg et al., 2009; Gilbert et al., 2010) and has been strongly linked to both the induction and propagation of cancer in humans (Mantovani et al., 2008).

The finding that certain FDA drugs inhibit Raf-1^{gof} TSCs but permit compensatory overproliferation of genetically wild-type stem cells demonstrates that Drosophila ISCs can be used to model different aspects of mammalian cell druggability. Raf-1^{gof} TSCs, like rapidly dividing mammalian cells, are susceptible to broadly cytotoxic drugs, including those that can have clinical relevance in humans, such as FDA anti-cancer drugs. On the other hand, overproliferating ISCs, like conventionally defined mammalian CSCs, are generally resistant to FDA drugs. Both ISC models may be used to find clinically relevant compounds. The rapidly dividing Raf-1^{gof} TSCs were sensitive not only to drugs already known to have clinical effectiveness in humans, but were also sensitive to pathway-specific drugs that target the core regulatory networks of stem cells, such as the MEK, mTOR, and Notch inhibitors identified in the present screen. The FDA drug-resistant overproliferating stem cells can be used in secondary screens to identify small molecules that overcome stem cell barriers to conventional FDA drugs.

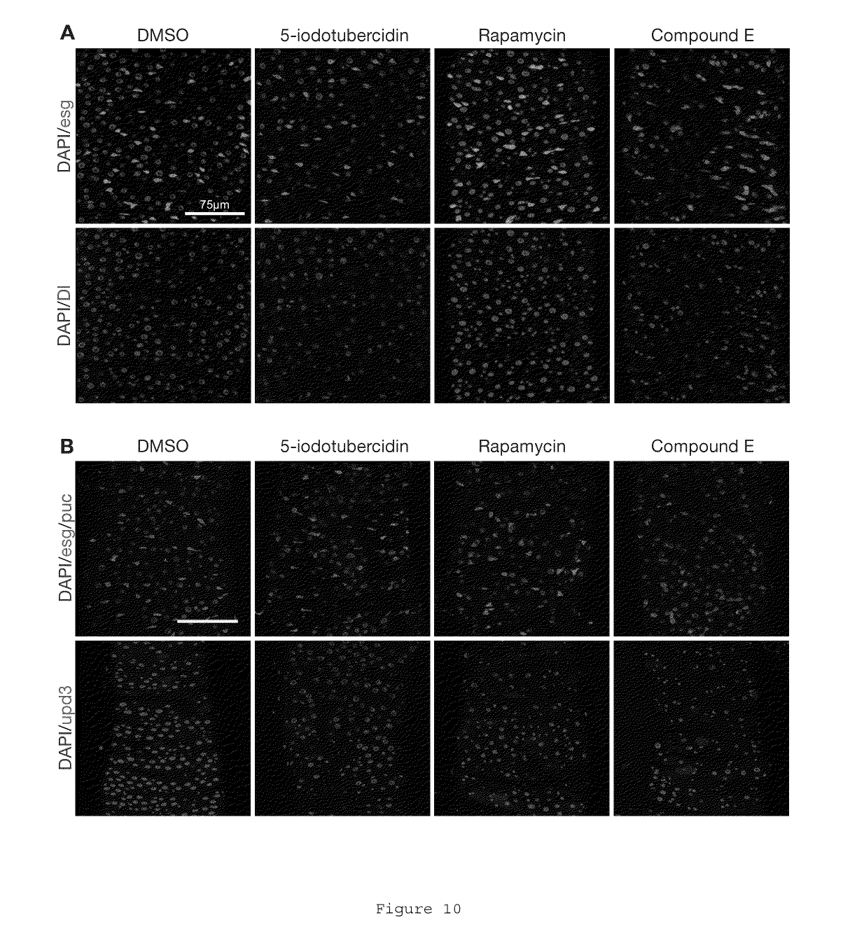

The present results show that several anti-cancer drugs can have a counterproductive effect on the microenvironment that ultimately drives stem cells to overproliferate. These observations invite new ways to think about cancer therapeutics. One approach, already established, is to avoid potential microenvironment responses altogether by using drugs that selectively target CSCs (Gupta et al., 2009), and not their differentiated daughter or “bulk tumor” cells. For example, the pathway-specific inhibitors that were identified, the MEK, mTOR, and Notch inhibitors, each target regulatory networks of the stem cells without affecting the microenvironment (FIG. 10). However, since daughter cells can be induced to revert to a CSC-like fate, at least experimentally (Gupta et al., 2009), successful treatments will likely require drugs that also target daughter cells. Indeed, a combinatorial approach using an anti-CSC drug, metformin, with a conventional FDA cytotoxic drug, doxorubicin, was found to be much more effective than use of either alone in a murine cancer model (Hirsch et al., 2009). The present results suggest that this combinatorial approach may be even more effective with the addition of drugs that suppress the growth-promoting responses from the microenvironment, such as JAK-STAT inhibitors. As the present results show, screens in Drosophila can uncover important molecules and mechanistic insights. In vivo models that preserve the stem cell microenvironment will prove indispensable in future discoveries of effective anti-CSC drugs and combinatorial therapeutics.

### REFERENCES

Agaisse et al., Dev. Cell, 5:441 (2003).
Amcheslaysky et al., Cell Stem Cell, 4:49 (2009).
Apidianakis et al., Proc. Natl. Acad. Sci. USA, 106:20883 (2009).
Barker et al., Genes Dev., 22:1856 (2008).
Barker et al., Nature, 457:608 (2009).
Biteau et al., Cell Stem Cell, 3:442 (2008).
Biteau et al., Development, 138:1045 (2011).
Brand et al., Genes Dev., 8:629 (1994).
Bromberg et al., Cancer Cell, 15:79 (2009).
Buchon et al., Genes Dev., 23:2333 (2009).
Casali et al., Cell Stem Cell, 4:124 (2009).
Caussinus et al., Nat. Genet., 37:1125 (2005).
Downward, Nat. Rev. Cancer, 3:11 (2003).
Fre et al., Nature, 435:964 (2005).
Gilbert et al., Cell, 143:355 (2010).
Gupta et al., Cell, 138:645 (2009).
Hirsch et al., Cancer Res., 69:7507 (2009).
Jacobsen et al., Development, 125:4531 (1998).
Jiang et al., Cell Stem Cell, 8:84 (2011).
Jiang et al., Cell, 137:1343 (2009).
Koch et al., Curr. Top. Dev. Biol., 92:411 (2010).
Lee et al., Development, 136:2255 (2009).
Lin et al., Nature, 455:1119 (2008).
Lobo et al., Annu Rev. Cell Dev. Biol., 23:675 (2007).
Mantovani et al., Nature, 454:436 (2008).
Markstein et al., Nat. Genet., 40:476 (2008).
Micchelli et al., Nature, 439:475 (2006).
Morrison et al., Cell 132:598 (2008).
Nateri et al., Nature, 437:281 (2005).
Ni et al., Nat. Methods, 5:49 (2008).
Ohlstein et al., Nature, 439:470 (2006).
Ohlstein et al., Science, 315:988 (2007).
Perrimon et al., Dev. Biol., 118:28 (1986).
Rajagopalan et al., Nature, 418:934 (2002).
Ring et al., Dev. Suppl., 251 (1993).
Stanger et al., Proc. Natl. Acad. Sci. USA, 102:12443 (2005).
Takashima et al., Nature, 454:651 (2008).
Van der Flier et al., Annu Rev. Physiol., published online 2008, 10.1146/annurev.physiol.010908.163145.
Voog et al., Cell Stem Cell, 6:103 (2010).
Zoncu et al., Nat. Rev. Mol. Cell. Biol., 12:21 (2011).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A transgenic Drosophila comprising a plurality of expression cassettes, which plurality comprises at least four of the following expression cassettes: a first expression cassette having a promoter operably linked to an open reading frame for a heterologous transcription factor; a second expression cassette having a transcriptional regulatory element comprising a nucleotide sequence that binds the transcription factor and a promoter operably linked to an oncogene; a third expression cassette having a transcriptional regulatory element comprising a nucleotide sequence that binds the transcription factor and a promoter operably linked to a first marker gene that encodes an optically detectable gene product; a fourth expression cassette having a promoter operably linked to an open reading frame encoding a gene product that regulates the heterologous transcription factor; and a fifth expression cassette having a transcriptional regulatory element comprising a nucleotide sequence that binds the transcription factor and a promoter operably linked to a second marker gene that encodes an optically detectable gene product.

2. The transgenic Drosophila of claim 1 wherein the first expression cassette has a gut-specific promoter operably linked to the open reading frame and the fourth expression cassette has a constitutive promoter operably linked to an open reading frame encoding a repressor of the transcription factor, which repressor is a conditional mutant.

3. The transgenic Drosophila of claim 1 which comprises all five expression cassettes.

4. The transgene Drosophila of claim 1 which comprises the first, third, fourth and fifth expression cassettes.

5. The transgenic Drosophila of claim 2 wherein the conditional mutant is a temperature sensitive mutant.

6. The transgenic Drosophila of claim 2 wherein the repressor represses the transcription factor at room temperature.

7. The transgenic Drosophila of claim 1 wherein one of the optically detectable gene products is a luminescent protein and the other is a fluorescent protein.

8. The transgenic Drosophila of claim 1 wherein one of the optically detectable markers is a luciferase.

9. The transgenic Drosophila of claim 1 wherein the oncogene is Raf^{gof}, myr-AKT or Notch^{DN}.

10. A method to screen compounds, comprising:
providing the transgenic Drosophila of claim 1 which expresses the transcription factor under conditions that allow for binding of the transcription factor to the nucleotide sequence;
exposing the transgenic Drosophila to one or more compounds; and
detecting at least one of the optically detectable gene products in or in a portion of the transgenic Drosophila exposed to the one or more compounds, wherein an alteration in the presence or amount of the at least one optically detectable gene product relative to the presence or amount of the corresponding optically detectable gene product in a corresponding control transgenic Drosophila that is not exposed to the one or more compounds or a portion of the control transgenic Drosophila, is indicative of a compound that alters the number of intestinal stem cells.

11. The method of claim 10 wherein the one or more compounds are ingested or inhaled by the transgenic Drosophila.

12. The method of claim 10 wherein the presence or amount of at least one of the optically detectable gene products is detected in the intestine.

13. The method of claim 10 wherein the presence or amount of at least one of the optically detectable gene products is detected in homogenates.

14. The method of claim 10 wherein one of the compounds decreases the number of intestinal tumor stem cells but not wild-type intestinal stem cells.

15. The method of claim 10 wherein one of the compounds enhances the number of wild-type intestinal stem cells.

16. The method of claim 10 wherein an increase in one of the optically detectable products is detected.

17. The method of claim 10 wherein a decrease in one of the optically detectable products is detected.

18. The method of claim 10 wherein the transgenic Drosophila is further exposed to an agent that activates the JNK-stress-related pathway and/or induces expression of IL-6 related cytokines.

19. The method of claim 10 wherein one of the detected compounds is a pathway-specific compound.

20. A method to selectively inhibit cancer stem cells in a mammal having refractory intestinal cancer or a relapse of intestinal cancer, comprising selecting a compound that alters the activity of intestinal tumor stem cells but not wild-type stem cells; and administering to the mammal an effective amount of the compound.
